(19) United States
(12) Patent Application Publication
(10) Pub. No.: US 2023/0406914 A1
(43) Pub. Date: Dec. 21, 2023

(54) METHODS FOR REDUCING HOST CELL PROTEIN CONTENT IN ANTIBODY PURIFICATION PROCESSES AND ANTIBODY COMPOSITIONS HAVING REDUCED HOST CELL PROTEIN CONTENT

(71) Applicant: Eli Lilly and Company, Indianapolis, IN (US)

(72) Inventors: Brian David BOWES, Indianapolis, IN (US); Lara Ellen KREBS, Indianapolis, IN (US); Lihua HUANG, Carmel, IN (US); Steven A. PLICHTA, Brownsburg, IN (US); Sarah M. RICHER, Avon, IN (US)

(21) Appl. No.: 18/247,198

(22) PCT Filed: Oct. 4, 2021
(86) PCT No.: PCT/US2021/053407

§ 371 Date: Mar. 29, 2023

Related U.S. Application Data
(60) Provisional application No. 63/086,915, filed on Oct. 2, 2020.

(51) Int. Cl. C07K 16/18

(52) U.S. Cl. CPC C07K 16/18; C07K 2317/10; C07K 2317/14; C07K 2317/76

(57) ABSTRACT

The present disclosure relates to methods for reducing host cell protein content in antibody preparation recombinantly produced in a host cell in the manufacturing process of antibodies intended for administration to a patient. The disclosed methods may be performed in order to prepare therapeutic antibody preparations having reduced host cell protein.

The present invention relates to the field of recombinant protein manufacturing. More particularly, the present invention provides a method for reducing host cell protein content in a protein preparation recombinantly produced in a host cell in the manufacturing process of proteins intended for administration to a patient, such as therapeutic or diagnostic antibodies or antigen-binding fragments thereof. The disclosed methods may be performed in order to produce antibody compositions having reduced host cell protein content.

Host Cell Proteins (HCPs) are proteins of the host cells that are involved in cell maintenance and growth, and protein synthesis and processing. However, in the realm of therapeutic or diagnostic proteins, the presence of HCPs threatens product quality and patient safety by posing concerns such as aggregation, product fragmentation by catalytic activity and/or immunogenicity. Hence, HCPs are identified as a critical quality attribute (CQA) of protein formulations. The formation of undesired aggregates and product fragmentation require additional purification steps to reduce/remove HCPs and these additional purification steps often result in reduced yield of the desired protein and increased overall manufacturing costs.

The challenges of eliminating HCPs from manufacturing processes and attempts to improve the processes to reduce HCPs have been disclosed, for example as set forth in Gilgunn et al; Goey et al., Biotechnology Advances 36 (2018) 1223-1237; and Current Opinion in Chemical Engineering 2018, 22:98-106. However, these processes to remove HCPs have limitations. For example, in some instances, these disclosures demonstrate one or more of, incomplete removal of HCPs, inconsistency in processes in removal of HCPs leading to aggregation, co-purification of the desired proteins and HCPs, impaired product function, immunogenicity concerns in patients, and/or reduced pharmacokinetic properties such as half-life. Furthermore, the processes developed to remove HCPs often require for example, the need to work with increased volumes and additional purification steps, often resulting in increased manufacturing costs and reduced yield. In some instances, the applicability of the method is limited to a specific molecule and/or format. As such, there remains a need for alternative methods of reducing HCPs in the purification process of therapeutic or diagnostic proteins. Such alternative methods reduce HCPs preferably without affecting product stability, yield, or cost to ultimately maintain product quality and is amenable to large scale manufacturing and ensuring patient safety.

Accordingly, the present invention addresses one or more of the above problems by providing alternative methods of reducing HCPs in the preparation of therapeutic or diagnostic antibodies or antigen-binding fragments thereof. The methods of the present invention provide reproducible methods that are highly effective in removing HCPs, whilst preserving antibody stability, reducing aggregation, maintaining product yield and have a potential to lower immunogenicity risk. Such methods can effectively remove HCPs without requiring increased antibody preparation volume. Surprisingly, the methods of the present invention achieved HCP counts well below the industry acceptable standards of <100 ppm. Surprisingly, other embodiments of the present invention achieved HCP counts of <50 ppm whilst preserving protein stability, reducing aggregation, and maintaining product yield. More surprisingly, other embodiments of the present invention achieved HCP counts of <20 ppm, <10 ppm, <5 ppm, <1 ppm, or ˜0 ppm, whilst preserving protein stability, reducing aggregation, and maintaining product yield. Furthermore, embodiments of the present invention provide methods of HCP removal that are applicable to a broad range of molecules. Other embodiments of the present invention enable the elimination of additional purification steps, resulting in a reduction in batch processing time, and decreased manufacturing costs. The disclosed methods may be performed in order to produce antibody compositions having reduced host cell content, wherein the host cell content of the antibody compositions is less <100 ppm, <50 ppm, <10 ppm, <5 ppm, <1 ppm, or ˜0 ppm.

Accordingly, there is provided methods of reducing host cell protein content in an anti-N3pGlu Aβ antibody (“an anti-N3pG antibody”) preparation. In some embodiments. the anti-N3pG antibody is recombinantly produced in a mammalian host cell, such as a Chinese hamster ovary cell host cell.

Accordingly, in particular embodiments, provided is a method of reducing host cell protein content in a protein preparation comprising an anti-N3pG antibody recombinantly produced in a mammalian host cell comprising, subjecting the protein preparation recombinantly produced in a host cell to an affinity chromatography column, eluting the anti-N3pGlu Aβ antibody from the chromatography column with a buffer comprising a combination of a weak acid and a strong acid, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 50 ppm, to less than about 20 ppm, to less than about 10 ppm, to less than about 5 ppm, or less than about 1 ppm.

Accordingly, in particular embodiments, provided is a method of reducing host cell protein content in a protein preparation comprising an anti-N3pGlu Aβ antibody recombinantly produced in a mammalian host cell comprising, subjecting the protein preparation recombinantly produced in a host cell to an affinity chromatography column, eluting the anti-N3pGlu Aβ antibody from the chromatography column with a buffer comprising a combination of a weak acid and a strong acid, performing viral inactivation, raising the pH of the eluate to about pH 5.0 or higher (e.g., about pH 6.0 or higher, or about pH 7.0 or higher), subjecting the eluate comprising the protein to a depth filter, and obtaining a filtered protein preparation comprising an comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 50 ppm, to less than about 20 ppm, to less than about 10 ppm, to less than about 5 ppm, or less than about 1 ppm.

Accordingly, in particular embodiments, provided is a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column, to below about pH 4.0, and wherein the eluate is maintained at below about pH 4.0 for about 0 minutes to about 180 minutes, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 50 ppm, to less than about 20 ppm, to less than about 10 ppm, to less than about 5 ppm, or less than about 1 ppm.

In some embodiments of the invention, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, wherein the concentration of the acetic acid is about 20 mM, and wherein the concentration of the phosphoric acid is about 5 mM to about 10 mM, adjusting the pH of the eluate comprising the anti-N3pGlu Aβ antibody from said step of eluting the anti-N3pGlu Aβ antibody from the chromatography column, to below about pH 4.0, and wherein the eluate is maintained at below about pH 4.0 for about 0 minutes about 180 minutes, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 50 ppm, to less than about 20 ppm, to less than about 10 ppm, to less than about 5 ppm, or less than about 1 ppm.

In some embodiments of the invention, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is lactic acid, wherein the concentration of the acetic acid is about 20 mM, and wherein the concentration of the lactic acid is about 5 mM, adjusting the pH of the eluate comprising the anti-N3pGlu Aβ antibody from said step of eluting the anti-N3pGlu Aβ antibody from the chromatography column, to below about pH 4.0, and wherein the eluate is maintained at below about pH 4.0 for about 0 minutes to about 180 minutes, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 50 ppm, to less than about 20 ppm, to less than about 10 ppm, to less than about 5 ppm, or less than about 1 ppm.

In some embodiments, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column, wherein said step of adjusting the pH of the eluate comprises adding about 20 mM HCl to the eluate, wherein the pH of the eluate is adjusted to about pH 3.3 to about pH 3.7, and wherein the eluate is maintained at about pH 3.3 to about pH 3.7 for about 0 minutes to about 180 minutes, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In some embodiments, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column, wherein said step of adjusting the pH of the eluate comprises adding about 20 mM HCl to the eluate, wherein the pH of the eluate is adjusted to about pH 3.5, and wherein the eluate is maintained at about pH 3.5 for about 0 minutes to about 180 minutes, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 or higher, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In some particular embodiments, the present disclosure provides a method of reducing host cell protein content in a protein preparation comprising an anti-N3pGlu Aβ antibody recombinantly produced in a host cell comprising, subjecting the protein preparation comprising an anti-N3pGlu Aβ antibody recombinantly produced in a host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column to below about pH 4.0, and wherein the eluate is maintained at below about pH 4.0 for about 0 minutes to about 180 minutes, raising the pH of the eluate to about pH 5.0 to about pH 7.5 comprising adding about 250 mM Tris Buffer to the eluate, and subjecting the eluate comprising the anti-N3pGlu Aβ antibody to a depth filter, and obtaining a filtered protein preparation comprising an anti-N3pGlu Aβ antibody. In some embodiments, raising the pH of the eluate to about pH 5.0 to about pH 7.5 comprises adding about 100 mM to about 1000 mM Tris Buffer to the eluate. In some embodiments the ionic strength of the eluate from the step of raising the pH to above about pH 5.0 to about pH 7.5, is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In some embodiments, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column to below about pH 4.0, and wherein the eluate is maintained at below about pH 4.0 for about 0 minutes to about 180 minutes, raising the pH of the eluate to about pH 7.0 comprising adding about 250 mM Tris buffer to the eluate, subjecting the eluate comprising the antibody to a depth filter, and obtaining a filtered antibody preparation. In some embodiments, raising the pH of the eluate to about pH 6.5 to about pH 7.5 (e.g. about pH 7.0) comprises adding about 100 mM to about 1000 mM Tris Buffer to the eluate. In some embodiments the ionic strength of the eluate from the step of raising the pH to about pH 6.5 to about pH 7.5 (e.g., about pH 7.0), is about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In some embodiments, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column to below about pH 4.0, and wherein the eluate is maintained at below about pH 4.0 for about 0 minutes to about 180 minutes, raising the pH of the eluate to about pH 5.0 or higher (e.g., about pH 6.0 or higher, or about pH 7.0 or higher), subjecting the eluate comprising the anti-N3pGlu Aβ antibody to a depth filter, and obtaining a filtered protein preparation comprising an anti-N3pGlu Aβ antibody, wherein the eluate subjected to the depth filter has an ionic strength of about 10 mM to about 45 mM. Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In particular embodiments, the present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid is acetic acid and the strong acid is phosphoric acid, or lactic acid, adjusting the pH of the eluate comprising the anti-N3pGlu Aβ antibody from said step of eluting the anti-N3pGlu Aβ antibody from the chromatography column to below about pH 4.0, and wherein the eluate is maintained at below about pH 4.0 for about 0 minutes to about 180 minutes and wherein viral inactivation is achieved.

The present disclosure provides a method of reducing host cell protein content in a protein preparation comprising an anti-N3pGlu Aβ antibody recombinantly produced in a mammalian host cell comprising, subjecting the protein preparation comprising an anti-N3pGlu Aβ antibody recombinantly produced in a mammalian host cell to an affinity chromatography column, eluting the anti-N3pGlu Aβ antibody from the chromatography column with a buffer comprising a combination of a weak acid and a strong acid, wherein the weak acid comprises acetic acid at a concentration of about 20 mM, and wherein the strong acid comprises of any one of phosphoric acid, formic acid, or lactic acid, and wherein the concentration of the strong acid is about 5 mM to about 10 mM, adjusting the pH of the eluate comprising the anti-N3pGlu Aβ antibody from said step of eluting the anti-N3pGlu Aβ antibody from the chromatography column, wherein said step of adjusting the pH of the eluate comprises adding any one of HCl, phosphoric acid, citric acid, acetic acid, or a combination thereof (e.g., a combination of acetic acid plus phosphoric acid or a combination of acetic acid and citric acid), to the eluate, wherein the pH is adjusted to below about pH 4.0, and wherein the eluate is maintained at below about pH 4.0 for about 0 minutes to about 180 minutes, raising the pH of the eluate to about pH 5.0 to about pH 7.5, subjecting the eluate comprising the anti-N3pGlu Aβ antibody to a depth filter, and obtaining a filtered protein preparation comprising an anti-N3pGlu Aβ antibody. In some embodiments the ionic strength of the eluate from the step of raising the pH to about pH 5.0 to about 7.5, is about 10 mM to about 45 mM.

Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In further embodiments, the elution step comprises an elution buffer comprising of a combination of any one of acetic acid and phosphoric acid, acetic acid and lactic acid, or acetic acid and formic acid, and wherein the step of adjusting the pH to below about pH 4.0 comprises adding any one of HCl, phosphoric acid, citric acid, acetic acid, or a combination thereof (e.g., a combination of acetic acid plus phosphoric acid or a combination of acetic acid and citric acid). In further embodiments, the elution step comprises an elution buffer comprising a combination of any one of about 20 mM acetic acid and about 10 mM phosphoric acid, about 20 mM acetic acid and about 5 mM phosphoric acid, or about 20 mM acetic acid and about 5 mM formic acid, and wherein the step of adjusting the pH to below about pH 4.0 comprises adding any one of about 20 mM HCl, about 15 mM to about 200 mM phosphoric acid, about 1000 mM citric acid, or a combination of about 20 mM acetic acid and about 10 mM phosphoric acid. In such embodiments the ionic strength of the eluate from the step of raising pH to above pH of about 6.0, is about 10 mM to about 45 mM.

In one aspect of the invention, the invention provides a method of reducing host cell protein content in a protein preparation comprising an anti-N3pGlu Aβ antibody recombinantly produced in a mammalian host cell, comprising the steps of:
subjecting the protein preparation comprising an anti-N3pGlu Aβ antibody recombinantly produced in a mammalian host cell to an affinity chromatography column;
eluting the anti-N3pGlu Aβ antibody from the chromatography column with a buffer comprising a combination of a weak acid and a strong acid; wherein the weak acid is acetic acid and the strong acid is phosphoric acid or lactic acid;
adjusting the pH of the eluate comprising the anti-N3pGlu Aβ antibody from said step of eluting the anti-N3pGlu Aβ antibody from the chromatography column, to below about pH 4.0, and wherein the eluate is maintained at below about pH 4.0 for about 0 minutes to about 180 minutes;
raising the pH of the eluate to about pH 5.0 or higher (e.g., about pH 6.0 or higher, or about pH 7.0 or higher);
subjecting the eluate comprising the anti-N3pGlu Aβ antibody to a depth filter, and
obtaining a filtered protein preparation comprising an anti-N3pGlu Aβ antibody.

Preferably, the host cell protein content in the protein preparation comprising an anti-N3pGlu Aβ antibody is reduced. More preferably, the host cell protein content in the protein preparation comprising an anti-N3pGlu Aβ antibody is reduced to less than about 100 ppm, to less than about 10 ppm, to less than about 5 ppm, or less than about 1 ppm.

In a further embodiment of the present invention, there is provided a method of reducing host cell protein content in a protein preparation comprising an anti-N3pGlu Aβ antibody recombinantly produced in a mammalian host cell, the method comprising the steps of:
a) subjecting the protein preparation to an affinity chromatography column;
b) eluting the anti-N3pGlu Aβ antibody from the chromatography column to obtain an eluate comprising the anti-N3pGlu Aβ antibody;
c) adjusting, if necessary, the pH of the eluate to between pH 5.0 and pH 7.5, subjecting the eluate to a depth filter and obtaining a filtered protein preparation comprising the anti-N3pGlu Aβ antibody, wherein the depth filter is a fully synthetic depth filter.

Preferably, the chromatography column comprises a Protein A, Protein G or Protein L affinity chromatography column. Further preferably, the depth filter pore size is at least from about 9p (micron) to about 0.1μ. Still further preferably, the depth filter pore size is from at least from about 2μ to about 0.1μ. Still further preferably, the depth filter pore size is about 0.1μ. Still further preferably, the depth filter is a X0SP filter. In an alternative embodiment of the present invention, the pH of the eluate on the depth filter is about 5.0. In a further alternative embodiment of the present invention, the pH of the eluate on the depth filter is about 6.0. In a further alternative embodiment of the present invention, the pH of the eluate on the depth filter is about 7.0.

This particular embodiment encompasses methods wherein the anti-N3pG antibody is eluted from the affinity chromatography column with any commonly used weak or strong acids, including but not limited to acetic acid, citric acid, phosphoric acid, hydrochloric acid, formic acid, and lactic acid.

It has been found that the use of a fully synthetic filter at a pH of the solution on the filter of 5.0 to 7.0 is quite effective at reducing and/or removing HCPs when compared to more traditional cellulose/diatomaceous earth-based filters.

The disclosed methods may be performed in order to reduce host cell proteins (HCPs) in a preparation comprising an anti-N3pGlu Aβ antibody or an antigen-binding fragment thereof in order to obtain an antibody composition having a reduced HCP content. In some embodiments, the anti-N3pGlu Aβ antibody is a monoclonal antibody, a chimeric antibody, a humanized antibody, a human antibody, a bispecific antibody, or an antibody fragment. In some embodiments, the anti-N3pGlu Aβ antibody is an IgG1 antibody or contains the Fc portion of an IgG1 antibody. Disclosed herein is an anti-SARS-COV-2 antibody.

In some embodiments of the disclosed methods and the compositions produced the disclosed methods, the anti-N3pG antibody is donanemab. In some embodiments, the anti-N3pG antibody comprises a light chain variable region (LH) comprising LH complementarity determining region 1 (LCDR1), LCDR2, and LCDR3 which are present in the amino acid sequence of

(SEQ ID NO: 13)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIK;

and the anti-N3pG antibody comprises a heavy chain variable region (VH) comprising VH complementarity determining region 1 (HCDR1), HCDR2 and HCDR3, which are present in the amino acid sequence

(SEQ ID NO: 14)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSS.

In some embodiments, the anti-N3pG antibody comprises an LCDR1 of

LCDR1 is
(SEQ ID NO: 17)
KSSQSLLYSRGKTYLN,
LCDR2 is
(SEQ ID NO: 18)
AVSKLDS,
LCDR3 is
(SEQ ID NO: 19)
VQGTHYPFT,
HCDR1 is
(SEQ ID NO: 20)
GYDFTRYYIN,
HCDR2 is
(SEQ ID NO: 21)
WINPGSGNTKYNEKFKG,
and
HCDR3 is
(SEQ ID NO: 22)
EGITVY.

In some embodiments, the anti-N3pG antibody comprises a variable light chain (LC) comprising of an amino acid sequence of

(SEQ ID NO: 13)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIK

and a variable heavy chain (HC) comprising of an amino acid sequence of

(SEQ ID NO: 14)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSS.

In some embodiments, the anti-N3pG antibody comprises a light chain (LC) comprising of an amino acid sequence of

(SEQ ID NO: 15)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

and a heavy chain (HC) comprising of an amino acid sequence of

(SEQ ID NO: 16)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPG.

In some embodiments, the anti-N3pG antibody comprises a light chain (LC) comprising an amino acid sequence encoded by the DNA sequence of

(SEQ ID NO: 33)
gatattgtgatgactcagactccactctccctgtccgtcacccctggac
agccggcctccatctcctgcaagtcaagtcagagcctcttatatagtcg
cggaaaaacctatttgaattggctcctgcagaagccaggccaatctcca
cagctcctaatttatgcggtgtctaaactggactctggggtcccagaca
gattcagcggcagtgggtcaggcacagatttcacactgaaaatcagcag
ggtggaggccgaagatgttggggtttattactgcgtgcaaggtacacat
tacccattcacgtttggccaagggaccaagctggagatcaaacgaactg
tggctgcaccatctgtcttcatcttcccgccatctgatgagcagttgaa
atctggaactgcctctgttgtgtgcctgctgaataacttctatcccaga
gaggccaaagtacagtggaaggtggataacgccctccaatcgggtaact
cccaggagagtgtcacagagcaggacagcaaggacagcacctacagcct
cagcagcaccctgacgctgagcaaagcagactacgagaaacacaaagtc
tacgcctgcgaagtcacccatcagggcctgagctcgcccgtcacaaaga
gcttcaacaggggagagtgc

and a heavy chain (HC) comprising an amino acid sequence encoded by the DNA sequence of

(SEQ ID NO: 34)
caggtgcagctggtgcagtctggggctgaggtgaagaagcctgggtcct
cagtgaaggtttcctgcaaggcatctggttacgacttcactagatacta
tataaactgggtgcgacaggcccctggacaagggcttgagtggatggga
tggattaatcctggaagcggtaatactaagtacaatgagaaattcaagg
gcagagtcaccattaccgcggacgaatccacgagcacagcctacatgga
gctgagcagcctgagatctgaggacacggccgtgtattactgtgcgaga
gaaggcatcacggtctactggggccaagggaccacggtcaccgtctcct
cagcctccaccaagggcccatcggtcttcccgctagcaccctcctccaa
gagcacctctgggggcacagcggccctgggctgcctggtcaaggactac
ttccccgaaccggtgacggtgtcgtggaactcaggcgccctgaccagcg
gcgtgcacaccttcccggctgtcctacagtcctcaggactctactccct
cagcagcgtggtgaccgtgccctccagcagcttgggcacccagacctac
atctgcaacgtgaatcacaagcccagcaacaccaaggggacaagaaagt
tgagcccaaatcttgtgacaaaactcacacatgcccaccgtgcccagca
cctgaactcctggggggaccgtcagtcttcctcttccccccaaaaccca
aggacaccctcatgatctcccggacccctgaggtcacatgcgtggtggt
ggacgtgagccacgaagaccctgaggtcaagttcaactggtacgtggac
ggcgtggaggtgcataatgccaagacaaagccgcgggaggagcagtaca
acagcacgtaccgtgtggtcagcgtcctcaccgtcctgcaccaggactg
gctgaatggcaaggagtacaagtgcaaggtctccaacaaagccctccca
gcccccatcgagaaaaccatctccaaagccaaagggcagccccgagaac
cacaggtgtacaccctgcccccatcccgggacgagctgaccaagaacca
ggtcagcctgacctgcctggtcaaaggcttctatcccagcgacatcgcc
gtggagtgggagagcaatgggcagccggagaacaactacaagaccacgc
cccccgtgctggactccgacggctccttcttcctctatagcaagctcac
cgtggacaagagcaggtggcagcaggggaacgtcttctcatgctccgtg
atgcatgaggctctgcacaaccactacacgcagaagagcctctccctgt
ctccgggt.

In some embodiments of the disclosed methods and the composition produced by the disclosed methods, the anti-N3pG antibody is the antibody referred to as “Antibody 201c” in U.S. Pat. No. 10,647,759, the content of which is incorporated herein by reference in its entirety. In some embodiments, the anti-N3pG antibody comprises a light chain variable region (LH) comprising LH complementarity determining region 1 (LCDR1), LCDR2, and LCDR3 which are present in the amino acid sequence of

(SEQ ID NO: 23)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIK;

and the anti-N3pG antibody comprises a heavy chain variable region (VH) comprising VH complementarity determining region 1 (HCDR1), HCDR2 and HCDR3, which are present in the amino acid sequence of

(SEQ ID NO: 24)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSS.

In some embodiments, the anti-N3pG antibody comprises an LCDR1 of

(SEQ ID NO: 27)
RASQSLGNWLA,
an LCDR2 of
(SEQ ID NO: 28)
YQASTLES,
an LCDR3 of
(SEQ ID NO: 29)
QHYKGSFWT,
an HCDR1 of
(SEQ ID NO: 30)
AASGFTFSSYPMS,
an HCDR2 of
(SEQ ID NO: 31)
AISGSGGSTYYADSVKG,
and
an HCDR3 of
(SEQ ID NO: 32)
AREGGSGSYYNGFDY.

In some embodiments, the anti-N3pG antibody comprises a variable light chain (VL) comprising of an amino acid sequence of

(SEQ ID NO: 23)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIK;

and a variable heavy chain (VH) comprising of an amino acid sequence of

(SEQ ID NO: 24)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSS.

In some embodiments, the anti-N3pG antibody comprises a light chain (LC) comprising of an amino acid sequence of

(SEQ ID NO: 25)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

and a heavy chain (HC) comprising of an amino acid sequence of

(SEQ ID NO: 26)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPG.

In some embodiments, the anti-N3pG antibody comprises a light chain (LC) comprising an amino acid sequence encoded by the DNA sequence of

(SEQ ID NO: 35)
gacatccagatgacccagtctccttccaccctgtctgcatctgtaggag
acagagtcaccatcacttgccgggccagtcagagtcttggtaactggtt
ggcctggtatcagcagaaaccagggaaagcccctaaactcctgatctat
caggcgtctactttagaatctggggtcccatcaagattcagcggcagtg
gatctgggacagagttcactctcaccatcagcagcctgcagcctgatga
ttttgcaacttattactgccaacattataaaggttctttttggacgttc
ggccaagggaccaaggtggaaatcaaacggaccgtggctgcaccatctg
tcttcatcttcccgccatctgatgagcagttgaaatctggaactgcctc
tgttgtgtgcctgctgaataacttctatcccagagaggccaaagtacag
tggaaggtggataacgccctccaatcgggtaactcccaggagagtgtca
cagagcaggacagcaaggacagcacctacagcctcagcagcaccctgac
gctgagcaaagcagactacgagaaacacaaagtctacgcctgcgaagtc
acccatcagggcctgagctcgcccgtcacaaagagcttcaacaggggag
agtgc

and a heavy chain (HC) comprising an amino acid sequence encoded by the DNA sequence of

(SEQ ID NO: 36)
gaggtgcagctgttggagtctgggggaggcttggtacagcctggggggt
ccctgagactctcctgtgcagcctctggattcacctttagcagctatcc
tatgagctgggtccgccaggctccagggaaggggctggagtgggtctca
gctattagtggtagtggtggtagcacatactacgcagactccgtgaagg
gccggttcaccatctccagagacaattccaagaacacgctgtatctgca
aatgaacagcctgagagccgaggacacggccgtatattactgtgcgaga
gaggggggctcagggagttattataacggctttgattattggggccagg
gaaccctggtcaccgtctcctcagcctccaccaagggcccatcggtctt
cccgctagcaccctcctccaagagcacctctgggggcacagcggccctg
ggctgcctggtcaaggactacttccccgaaccggtgacggtgtcgtgga
actcaggcgccctgaccagcggcgtgcacaccttcccggctgtcctaca
gtcctcaggactctactccctcagcagcgtggtgaccgtgccctccagc
agcttgggcacccagacctacatctgcaacgtgaatcacaagcccagca
acaccaaggtggacaagaaagttgagcccaaatcttgtgacaaaactca
cacatgcccaccgtgcccagcacctgaactcctggggggaccgtcagtc
ttcctcttccccccaaaacccaaggacaccctcatgatctcccggaccc
ctgaggtcacatgcgtggtggtggacgtgagccacgaagaccctgaggt
caagttcaactggtacgtggacggcgtggaggtgcataatgccaagaca
aagccgcgggaggagcagtacaacagcacgtaccgtgtggtcagcgtcc
tcaccgtcctgcaccaggactggctgaatggcaaggagtacaagtgcaa
ggtctccaacaaagccctcccagcccccatcgagaaaaccatctccaaa
gccaaagggcagccccgagaaccacaggtgtacaccctgcccccatccc
gggacgagctgaccaagaaccaggtcagcctgacctgcctggtcaaagg
cttctatcccagcgacatcgccgtggagtgggagagcaatgggcagccg
gagaacaactacaagaccacgccccccgtgctggactccgacggctcct
tcttcctctatagcaagctcaccgtggacaagagcaggtggcagcaggg
gaacgtcttctcatgctccgtgatgcatgaggctctgcacaaccactac
acgcagaagagcctctccctgtctccgggt.

In another aspect of the invention, the invention provides a method of reducing host cell protein content in an anti-N3pG antibody preparation recombinantly produced in a host cell comprising the steps of:
subjecting the anti-N3pG antibody preparation recombinantly produced in a host cell to an affinity chromatography column, e.g., a Protein A affinity chromatography column;
eluting the anti-N3pG antibody with a buffer comprising a combination of acetic acid and phosphoric acid, or a combination of acetic acid and lactic acid;
adjusting the pH of the eluate comprising the anti-N3pG antibody by addition of about 20 mM HCl, wherein the pH is adjusted to about pH 3.3 to about pH 3.7, and wherein the eluate is maintained at about pH 3.3 to about pH 3.7 for about 0 minutes to about 180 minutes;
raising the pH of the eluate comprising the anti-N3pG antibody by addition of about 250 mM Tris Buffer, wherein the pH is raised to about pH 5.0 to about pH 7.5;
subjecting the eluate comprising the anti-N3pG antibody to a depth filter, and obtaining a filtered anti-N3pG antibody preparation,
wherein host cell protein content in the anti-N3pG antibody preparation after depth filtration is reduced to less than about 100 ppm, 50 ppm, 20 ppm, 10 ppm, 5 ppm, or 1 ppm, and wherein the anti-N3pG antibody is an IgG1 antibody.

In some embodiments of the invention, the present disclosure provides a method of reducing host cell protein content in an anti-N3pG antibody preparation recombinantly produced in a host cell comprising, subjecting the anti-N3pG antibody preparation recombinantly produced in a host cell to a Protein A chromatography column, eluting the anti-N3pG antibody from the chromatography column with a buffer comprising a combination of about 20 mM acetic acid and about 5 mM phosphoric acid, or a buffer comprising a combination of about 20 mM acetic acid and about 10 mM phosphoric acid, or a buffer comprising a combination of about 20 mM acetic acid and about 5 mM lactic acid, adjusting the pH of the eluate comprising the anti-N3pG antibody by addition of about 20 mM HCl, wherein the pH is lowered to about pH 3.3 to about pH 3.7, and wherein the eluate is maintained at about pH 3.3 to about pH 3.7 for about 0 minutes to about 180 minutes, raising the pH of the eluate comprising the anti-N3pG antibody by addition of about 250 mM Tris Buffer, wherein the pH is raised to about pH 5.0 to about pH 7.5, subjecting the eluate comprising the anti-N3pG antibody to a depth filter, and obtaining a filtered anti-N3pG antibody preparation, wherein the host cell protein content in the filtered anti-N3pG antibody preparation is less than about 100 ppm, 50 ppm, 20 ppm, 10 ppm, 5 ppm, or 1 ppm, and wherein the anti-N3pG antibody is an IgG1 antibody. In some embodiments, raising the pH of the eluate to about pH 5.0 to about pH 7.5 comprises adding about 100 mM to about 1000 mM Tris Buffer to the eluate.

In some embodiments of the invention, the present disclosure provides a method of reducing host cell protein content in anti-N3pG antibody preparation recombinantly produced in a host cell comprising, subjecting the anti-N3pG antibody preparation recombinantly produced in a host cell to a Protein A chromatography column, eluting the anti-N3pG antibody from the chromatography column with a buffer comprising a combination of about 20 mM acetic acid and about 5 mM phosphoric acid, or a buffer comprising a combination of about 20 mM acetic acid and about 10 mM phosphoric acid, or a buffer comprising a combination of about 20 mM acetic acid and about 5 mM lactic acid, adjusting the pH of the eluate comprising the anti-N3pG antibody with about 20 mM HCl, wherein the pH is adjusted to about pH 3.5, and wherein the eluate is maintained at about pH 3.5 for about 0 minutes to about 180 minutes, raising the pH of the eluate comprising the anti-N3pG antibody with about 250 mM Tris Buffer, wherein the pH is raised to about pH 5.0 to about pH 7.5, subjecting the eluate comprising the anti-N3pG antibody to a depth filter, and obtaining a filtered anti-N3pG antibody preparation, wherein the host cell protein content in the filtered anti-N3pG antibody is about less than about 100 ppm, 50 ppm, 20 ppm, 10 ppm, 5 ppm, or 1 ppm, and wherein the anti-N3pG antibody is an IgG1 antibody. In some embodiments, raising the pH of the eluate to about pH 5.0 to about pH 7.5 comprises adding about 100 mM to about 1000 mM Tris Buffer to the eluate.

In some embodiments of the invention, the present disclosure provides a method of reducing host cell protein content in an anti-N3pG antibody preparation recombinantly produced in a host cell comprising, subjecting the anti-N3pG antibody preparation recombinantly produced in a mammalian host cell to a Protein A chromatography column, eluting the anti-N3pG antibody from the chromatography column with a buffer comprising a combination of about 20 mM acetic acid and about 5 mM phosphoric acid, or a buffer comprising a combination of about 20 mM acetic acid and about 10 mM phosphoric acid, or a buffer comprising a combination of about 20 mM acetic acid and about 5 mM lactic acid, adjusting the pH of the eluate comprising the anti-N3pG antibody by addition of about 20 mM HCl, wherein the pH is lowered to about pH 3.5, and wherein the eluate is maintained at about pH 3.5 for about 0 minutes to about 180 minutes, and wherein viral inactivation is achieved.

In some embodiments of the invention, the present disclosure provides a method of reducing host cell protein content in an anti-N3pG antibody preparation recombinantly produced in a host cell comprising, subjecting the anti-N3pG antibody preparation recombinantly produced in a host cell to a Protein A chromatography column, eluting the anti-N3pG antibody from the chromatography column with a buffer comprising a combination of about 20 mM acetic acid and about 5 mM phosphoric acid, or a buffer comprising a combination of about 20 mM acetic acid and about 10 mM phosphoric acid, or a buffer comprising a combination of about 20 mM acetic acid and about 5 mM lactic acid, adjusting the pH of the eluate comprising the anti-N3pG antibody by addition of about 20 mM HCl, wherein the pH is lowered to about pH 3.3 to about pH 3.7, and wherein the eluate maintained at about pH 3.3 to about pH 3.7 for about 0 minutes to about 180 minutes, raising the pH of the eluate comprising the anti-N3pG antibody with about 250 mM Tris Buffer, wherein the pH is raised to about pH 7.25, subjecting the eluate comprising the anti-N3pG antibody to a depth filter, and obtaining a filtered anti-N3pG antibody preparation, wherein the host cell protein content in the anti-N3pG antibody preparation is less than about 100 ppm, 50 ppm, 20 ppm, 10 ppm, 5 ppm, or 1 ppm, and wherein the anti-N3pG antibody is an IgG1 antibody. In some embodiments, raising the pH of the eluate to about pH 7.25 comprises adding about 100 mM to about 1000 mM Tris Buffer to the eluate.

In some embodiments of the invention, the present disclosure provides a method of reducing host cell protein content in an anti-N3pG antibody preparation recombinantly produced in a host cell comprising, subjecting the anti-N3pG antibody preparation recombinantly produced in a host cell to a Protein A chromatography column, eluting the anti-N3pG antibody from the chromatography column with a buffer comprising a combination of about 20 mM acetic acid and about 5 mM phosphoric acid, or a buffer comprising a combination of about 20 mM acetic acid and about 5 mM phosphoric acid, or a buffer comprising a combination of about 20 mM acetic acid and about 5 mM lactic acid, adjusting the pH of the eluate comprising the anti-N3pG antibody by addition of about 20 mM HCl, wherein the pH is lowered to about pH 3.5, and wherein the eluate is maintained at about pH 3.5 for about 0 minutes to about 180 minutes, raising the pH of the eluate comprising the anti-N3pG antibody by addition of about 250 mM Tris Buffer, wherein the pH is raised to about pH 7.25, subjecting the eluate comprising the anti-N3pG antibody to a depth filter, and obtaining a filtered anti-N3pG antibody preparation, wherein the host cell protein content in the anti-N3pG antibody preparation is less than about 100 ppm, 50 ppm, 20 ppm, 10 ppm, 5 ppm, or 1 ppm, and wherein the anti-N3pG antibody is an IgG1 antibody. In some embodiments, raising the pH of the eluate to about pH 7.25 comprises adding about 100 mM to about 1000 mM Tris Buffer to the eluate.

In some embodiments, the invention provides methods of reducing host cell protein content in an anti-N3pG antibody preparation recombinantly produced in a host cell,

In some embodiments of the disclosed methods and antibody compositions produced by the disclosed methods, the antibody is an antibody against the spike protein of sudden acute respiratory syndrome coronavirus 2 (SARS-CoV-2). In some embodiments, the anti-SARS-CoV-2 antibody is recombinantly produced in a mammalian host cell, such as a Chinese hamster ovary cell. Suitable anti-SARS-CoV-2 antibodies may include, but are not limited to, bamlanivimab, etesevimab, and bebtelovimab. In some embodiments, the anti-SARS-CoV-2 antibody is bamlanivimab. In some embodiments, the anti-SARS-COV-2 antibody comprises a variable heavy chain (VH) comprising of an amino acid sequence of SEQ ID NO: 1 and a variable light chain (VL) comprising of an amino acid sequence of SEQ ID NO: 2. In some embodiments, the anti-SARS-COV-2 antibody comprises a heavy chain (HC) comprising of an amino acid sequence of SEQ ID NO: 3 and a light chain (LC) comprising of an amino acid sequence of SEQ ID NO: 4. In other embodiments, the anti-SARS-COV-2 antibody is etesevimab. In yet other embodiments, the anti-SARS-COV-2 antibody comprises a variable heavy chain (VH) comprising of an amino acid sequence of SEQ ID NO: 5 and a variable light chain (VL) comprising of an amino acid sequence of SEQ ID NO: 6. In yet further embodiments, the anti-SARS-COV-2 antibody comprises a heavy chain (HC) comprising of an amino acid sequence of SEQ ID NO: 7 and a light chain (LC) comprising of an amino acid sequence of SEQ ID NO: 8. In some embodiments, the anti-SARS-COV-2 antibody is bebtelovimab. In yet other embodiments, the anti-SARS-COV-2 antibody comprises a variable heavy chain (VH) comprising of an amino acid sequence of SEQ ID NO: 9 and a variable light chain (VL) comprising of an amino acid sequence of SEQ ID NO: 10. In yet further embodiments, the anti-SARS-COV-2 antibody comprises a heavy chain (HC) comprising of an amino acid sequence of SEQ ID NO: 11 and a light chain (LC) comprising of an amino acid sequence of SEQ ID NO: 12.

In some embodiments, the therapeutic or diagnostic antibody, is produced in mammalian cells. In some embodiments, the mammalian cell is a Chinese Hamster Ovary (CHO) cells, or baby hamster kidney (BHK) cells, murine hybridoma cells, or murine myeloma cells.

In some embodiments, the invention provides methods wherein the method of reducing host cell protein content in an antibody preparation recombinantly produced in a host cell after subjecting to a depth filter is further subjected to further purification and/or polishing steps to obtain a drug substance preparation. Drug substance is defined by the FDA as an active ingredient that is intended to furnish pharmacological activity or other direct effect in the diagnosis, cure, mitigation, treatment, or prevention of disease or to affect the structure or any function of the human body but does not include intermediates used in the synthesis of such ingredient. Drug product is a finished dosage form that is suitable for administration to human patients, e.g., tablet, capsule, or solution, that contains a drug substance, generally, but not necessarily, in association with one or more other ingredients. In some embodiments, the further purification and/or polishing step comprises one or more of the following: performing viral inactivation, performing ion exchange chromatography, performing viral filtration, and/or performing tangential flow filtration.

In some embodiments, the present disclosure provides a method of reducing host cell protein content in a protein preparation comprising an anti-N3pG antibody recombinantly produced in a mammalian host cell, wherein the host cell protein content in the protein preparation comprising an anti-N3pG antibody is reduced to less than about 100 ppm. In other embodiments the host cell protein content in the protein preparation comprising an anti-N3pG antibody is reduced to less than about 50 ppm. In other embodiments the host cell protein content in the protein preparation comprising an anti-N3pG antibody is reduced to less than about 20 ppm. In other embodiments the host cell protein content in the protein preparation comprising an anti-N3pG antibody is reduced to less than about 10 ppm, 5 ppm, or 1 ppm. In other embodiments the host cell protein content in the protein preparation comprising an anti-N3pG antibody is reduced to about 0 ppm.

In some embodiments, the present disclosure provides a method of reducing host cell protein content in a protein preparation comprising an anti-N3pG antibody recombinantly produced in a mammalian host cell, wherein the host cell protein content in the protein preparation comprises PLBL2, and wherein the PLBL2 content is reduced to less than about 100 ppm. In other embodiments the PLBL2 content is reduced to less than about 50 ppm. In other embodiments the PLBL2 content is reduced to less than about 20 ppm. In other embodiments the PLBL2 content is reduced to less than about 10 ppm, 5 ppm, or 1 ppm. In other embodiments the PLBL2 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises lysosomal protective protein, and wherein the lysosomal protective protein content is reduced to less than about 100 ppm. In other embodiments the lysosomal protective protein content is reduced to less than about 50 ppm. In other embodiments the lysosomal protective protein content is reduced to less than about 20 ppm. In other embodiments the lysosomal protective protein content is reduced to less than about 10 ppm, 5 ppm, or 1 ppm. In other embodiments the lysosomal protective protein content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises protein S100-A6, and wherein the protein S100-A6 content is reduced to less than about 100 ppm. In other embodiments the protein S100-A6 content is reduced to less than about 50 ppm. In other embodiments the protein S100-A6 content is reduced to less than about 20 ppm. In other embodiments the protein S100-A6 content is reduced to less than about 10 ppm, 5 ppm, or 1 ppm. In other embodiments the protein S100-A6 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises protein S100-A11, and wherein the protein S100-A11 content is reduced to less than about 100 ppm. In other embodiments the protein S100-A11 content is reduced to less than about 50 ppm. In other embodiments the protein S100-A11 protein content is reduced to less than about 20 ppm. In other embodiments the protein S100-A11 content is reduced to less than about 10 ppm, 5 ppm, or 1 ppm. In other embodiments the protein S100-A11 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises ubiquitin-40S ribosomal protein S27a, and wherein the ubiquitin-40S ribosomal protein S27a content is reduced to less than about 100 ppm. In other embodiments the ubiquitin-40S ribosomal protein S27a content is reduced to less than about 50 ppm. In other embodiments the ubiquitin-40S ribosomal protein S27a content is reduced to less than about 20 ppm. In other embodiments the ubiquitin-40S ribosomal protein S27a content is reduced to less than about 10 ppm, 5 ppm, or 1 ppm. In other embodiments the ubiquitin-40S ribosomal protein S27a content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises kallikrein-11, and wherein the kallikrein-11 content is reduced to less than about 100 ppm. In other embodiments the kallikrein-11 content is reduced to less than about 50 ppm. In other embodiments the kallikrein-11 content is reduced to less than about 20 ppm. In other embodiments the kallikrein-11 content is reduced to less than about 10 ppm, 5 ppm, or 1 ppm. In other embodiments the kallikrein-11 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises serine protease HTRA1 isoform X1, and wherein the serine protease HTRA1 isoform X1 content is reduced to less than about 100 ppm. In other embodiments the serine protease HTRA1 isoform X1 content is reduced to less than about 50 ppm. In other embodiments the serine protease HTRA1 isoform X1 content is reduced to less than about 20 ppm. In other embodiments the serine protease HTRA1 isoform X1 content is reduced to less than about 10 ppm, 5 ppm, or 1 ppm. In other embodiments the serine protease HTRA1 isoform X1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises complement C1r subcomponent, and wherein the complement C1r subcomponent content is reduced to less than about 100 ppm. In other embodiments the complement C1r subcomponent content is reduced to less than about 50 ppm. In other embodiments the complement C1r subcomponent content is reduced to less than about 20 ppm. In other embodiments the complement C1r subcomponent content is reduced to less than about 10 ppm, 5 ppm, or 1 ppm. In other embodiments the complement C1r subcomponent content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises actin, aortic smooth muscle isoform X1, and wherein the actin, aortic smooth muscle isoform X1 content is reduced to less than about 100 ppm. In other embodiments the actin, aortic smooth muscle isoform X1 content is reduced to less than about 50 ppm. In other embodiments the actin, aortic smooth muscle isoform X1 content is reduced to less than about 20 ppm. In other embodiments the actin, aortic smooth muscle isoform X1 content is reduced to less than about 10 ppm, 5 ppm, or 1 ppm. In other embodiments the actin, aortic smooth muscle isoform X1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises heat shock cognate 71 kDa protein, and wherein the heat shock cognate 71 kDa protein content is reduced to less than about 100 ppm. In other embodiments the heat shock cognate 71 kDa protein content is reduced to less than about 50 ppm. In other embodiments the heat shock cognate 71 kDa protein content is reduced to less than about 20 ppm. In other embodiments the heat shock cognate 71 kDa protein content is reduced to less than about 10 ppm, 5 ppm, or 1 ppm. In other embodiments the heat shock cognate 71 kDa protein content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises polyubiquitin, and wherein the polyubiquitin content is reduced to less than about 100 ppm. In other embodiments the polyubiquitin content is reduced to less than about 50 ppm. In other embodiments the polyubiquitin content is reduced to less than about 20 ppm. In other embodiments the polyubiquitin content is reduced to less than about 10 ppm, 5 ppm, or 1 ppm. In other embodiments the polyubiquitin content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises peroxiredoxin-1, and wherein the peroxiredoxin-1 content is reduced to less than about 100 ppm. In other embodiments the peroxiredoxin-1 content is reduced to less than about 50 ppm. In other embodiments the peroxiredoxin-1 content is reduced to less than about 20 ppm. In other embodiments the peroxiredoxin-1 content is reduced to less than about 10 ppm, 5 ppm, or 1 ppm. In other embodiments the peroxiredoxin-1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises glutathione S-transferase Y1, and wherein the glutathione S-transferase Y1 content is reduced to less than about 100 ppm. In other embodiments the glutathione S-transferase Y1 content is reduced to less than about 50 ppm. In other embodiments the glutathione S-transferase Y1 content is reduced to less than about 20 ppm. In other embodiments the glutathione S-transferase Y1 content is reduced to less than about 10 ppm, 5 ppm, or 1 ppm. In other embodiments the glutathione S-transferase Y1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises 40S ribosomal protein S28, and wherein the 40S ribosomal protein S28 content is reduced to less than about 100 ppm. In other embodiments the 40S ribosomal protein S28 content is reduced to less than about 50 ppm. In other embodiments the 40S ribosomal protein S28 content is reduced to less than about 20 ppm. In other embodiments the 40S ribosomal protein S28 content is reduced to less than about 10 ppm, 5 ppm, or 1 ppm. In other embodiments the 40S ribosomal protein S28 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises thioredoxin isoform X1, and wherein the thioredoxin isoform X1 content is reduced to less than about 100 ppm. In other embodiments the thioredoxin isoform X1 content is reduced to less than about 50 ppm. In other embodiments the thioredoxin isoform X1 content is reduced to less than about 20 ppm. In other embodiments the thioredoxin isoform X1 content is reduced to less than about 10 ppm, 5 ppm, or 1 ppm. In other embodiments the thioredoxin isoform X1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises basement membrane-specific heparan sulfate proteoglycan core protein isoform X1, and wherein the basement membrane-specific heparan sulfate proteoglycan core protein isoform X1 content is reduced to less than about 100 ppm. In other embodiments the basement membrane-specific heparan sulfate proteoglycan core protein isoform X1 content is reduced to less than about 50 ppm. In other embodiments the basement membrane-specific heparan sulfate proteoglycan core protein isoform X1 content is reduced to less than about 20 ppm. In other embodiments the basement membrane-specific heparan sulfate proteoglycan core protein isoform X1 content is reduced to less than about 10 ppm, 5 ppm, or 1 ppm. In other embodiments the basement membrane-specific heparan sulfate proteoglycan core protein isoform X1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises tubulointerstitial nephritis antigen-like protein, and wherein the tubulointerstitial nephritis antigen-like protein content is reduced to less than about 100 ppm. In other embodiments the tubulointerstitial nephritis antigen-like protein content is reduced to less than about 50 ppm. In other embodiments the tubulointerstitial nephritis antigen-like protein content is reduced to less than about 20 ppm. In other embodiments the tubulointerstitial nephritis antigen-like protein content is reduced to less than about 10 ppm, 5 ppm, or 1 ppm. In other embodiments the tubulointerstitial nephritis antigen-like protein content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises galectin-1, and wherein the galectin-1 content is reduced to less than about 100 ppm. In other embodiments the galectin-1 content is reduced to less than about 50 ppm. In other embodiments the galectin-1 content is reduced to less than about 20 ppm. In other embodiments the galectin-1 content is reduced to less than about 10 ppm, 5 ppm, or 1 ppm. In other embodiments the galectin-1 content is reduced to about 0 ppm.

In some embodiments, the present disclosure provides a method of reducing host cell protein content in a protein preparation comprising an anti-N3pG antibody recombinantly produced in a host cell, wherein the host cell protein content in the protein preparation comprises cornifin alpha, and wherein the cornifin alpha content is reduced to less than about 100 ppm. In other embodiments the cornifin alpha content is reduced to less than about 50 ppm. In other embodiments the cornifin alpha content is reduced to less than about 20 ppm. In other embodiments the cornifin alpha content is reduced to less than about 10 ppm, 5 ppm, or 1 ppm. In other embodiments the cornifin alpha content is reduced to about 0 ppm.

In some embodiments the present invention provides methods of reducing host 5 cell protein content a protein preparation comprising an anti-N3pG antibody recombinantly produced in a host cell, wherein the protein preparation is subjected to depth filtration. In some embodiments, the protein preparation comprising an anti-N3pG antibody is subjected to a depth filter wherein the depth filter is one or more of a B1HC filter, a X0SP filter, a C0SP filter, a X0HC filter, an Emphaze™ AEX Hybrid Purifier filter, or a Zeta Plus (ZB Media) filter (such as, a Zeta Plus (60ZB05A) filter, a Zeta Plus (90ZB05A) filter, or a Zeta Plus (90ZB08A) filter), or a depth filter that has the same performance characteristics as any of a B1HC filter, a X0SP filter, a C0SP filter, a X0HC filter, an Emphaze™ AEX Hybrid Purifier filter, or a Zeta Plus (ZB Media) filter (such as, a Zeta Plus (60ZB05A) filter, a Zeta Plus (90ZB05A) filter, or a Zeta Plus (90ZB08A) filter).

In some embodiments, the a protein preparation comprising an anti-N3pG antibody is subjected to a depth filter wherein the depth filter is one or more of a B1HC filter, a X0HC filter, or a Zeta Plus (ZB Media) filter (such as, a Zeta Plus (60ZB05A) filter, a Zeta Plus (90ZB05A) filter, or a Zeta Plus (90ZB08A) filter), or a depth filter that has the same performance characteristics as any of a B1HC filter, a X0HC filter, or a Zeta Plus (ZB Media) filter (such as, a Zeta Plus (60ZB05A) filter, a Zeta Plus (90ZB05A) filter, or a Zeta Plus (90ZB08A) filter).

In some embodiments, the protein preparation comprising an anti-N3pG antibody is subjected to a depth filter wherein the depth filter is one or more of a X0SP filter, a C0SP filter, a X0HC filter, or an Emphaze™ AEX Hybrid Purifier filter, or a depth filter that has the same performance characteristics as any of a X0SP filter, a C0SP filter, or an Emphaze™ AEX Hybrid Purifier filter.

In some embodiments of the disclosed methods, the depth filter utilized in the methods is a fully synthetic depth filter comprising a fully synthetic filter media. In some embodiments, the depth filter pore size is from about 9 microns to about 0.1 microns. In some embodiments, the depth filter pore size is from about 2 microns to about 0.1 microns. In some embodiments, the depth filter pore size is about 0.1 microns.

In some embodiments of the disclosed methods, the pH of the protein preparation comprising an anti-N3pG antibody that is subjected to depth filtration is about 5.0, and/or 5 the pH of the eluate comprising the anti-N3pG antibody after depth filtration is about 5.0. In other embodiments, the pH of the protein preparation comprising an anti-N3pG antibody that is subjected to depth filtration is about 6.0, and/or the pH of the eluate comprising the anti-N3pG antibody after depth filtration is about 6.0. In other embodiments, the pH of the protein preparation comprising an anti-N3pG antibody that is subjected to depth filtration is about 7.0, and/or the pH of the eluate comprising the anti-N3pG antibody after depth filtration is about 7.0.

In some embodiments the present disclosure provides a method of reducing host cell protein content in a protein preparation comprising an anti-N3pG antibody recombinantly produced in a mammalian host cell, wherein the ionic strength of the eluate from the step of raising pH to about 5.0 or higher (e.g., to about 6.0 or to about 7.0), is about 10 mM to about 45 mM. In some embodiments, the ionic strength is less than about 30 mM. In some embodiments, the ionic strength is less than about 20 mM. In other embodiments the ionic strength is less than about 15 mM.

In some embodiments the invention provides methods wherein the protein preparation comprising an anti-N3pG antibody recombinantly produced in a mammalian host cell is subjected to a chromatography column. In some embodiments, the chromatography column is one or more of an affinity column, an ion exchange column, a hydrophobic interaction column, a hydroxyapatite column, or a mixed mode column. In some embodiments, the affinity chromatography column is a Protein A column, a Protein G column or a protein L column. In other embodiments, the ion exchange chromatography column is an anion exchange column or a cation exchange column. In some embodiments, the invention provides methods wherein the HCPs are sufficiently removed from the final product.

In some embodiments, the invention provides methods of reducing host cell protein content in a protein preparation comprising an anti-N3pG antibody recombinantly produced in a host cell, wherein the anti-N3pG antibody is a therapeutic or diagnostic antibody. In further embodiments, the therapeutic or diagnostic anti-N3pG antibody is a monoclonal antibody, a chimeric antibody, a humanized antibody, a human antibody, a bispecific antibody, or an antibody fragment.

In another aspect, provided herein are pharmaceutical compositions comprising the protein preparation comprising an anti-N3pG antibody. In further aspects the present disclosure provides a composition produced by the methods as described herein. In yet other embodiments the present disclosure provides a composition produced by the methods as described herein, wherein the host cell protein content in the composition is less than about 100 ppm, 50 ppm, 20 ppm, 10 ppm, 5 ppm, or 1 ppm.

The term “Host cell proteins” (HCPs) are proteins of the host cells that are involved in cell maintenance and growth, and protein synthesis and processing. Certain HCPs have been associated with immunogenicity concerns in patients and there is a desire by regulators to reduce HCPs in order to minimize immunogenicity concerns. One powerful technique for immunogenicity analysis relies on immunoinformatics tools, which have been shown to make reliable predictions useful for and validated within the design of both biotherapeutics and vaccines. Of particular relevance to HCP-driven immunogenicity is the T cell pathway, in which an antigen-presenting cell processes a foreign protein into constituent peptides, some of which (the “epitopes”) are recognized by major histocompatibility complex (MHC) class II proteins and brought to the cell surface for inspection by T cells. The formation of a ternary MHC: epitope: T cell receptor complex drives the initial naïve response and can stimulate subsequent B cell activation and maturation. Thus, much immunoinformatics research has been directed toward highly-reliable prediction of putative T cell epitopes (De Groot and Martin, Clin Immunol. 2009 May; 131(2):189-20P1, which is hereby incorporated by reference in its entirety), and the EpiMatrix system is one heavily validated method based on peptide: MHC binding profiles. In addition to identifying individual epitopes within a protein, EpiMatrix can then also assess the overall immunogenicity risk of a protein according to its epitope density relative to benchmark proteins (De Groot and Martin, 2009). A general rule of thumb when using the EpiMatrix tool to predict immunogenicity is that those with a score of +20 and above carry an elevated immunogenicity risk and it is therefore desirable to reduce or eliminate such HCPs from the final preparation.

Such HCPs for example include those from Chinese Hamster Ovary (CHO) cells, e.g., Phospholipase B-like 2 protein (PLBL2) (GenBank Accession No. 354497505), S100-A6 (GenBank Accession No. 354478978), protein 5100-A11 (GenBank Accession No. 354490016), lysosomal protective protein (GenBank Accession No. 354476738), ubiquitin-40S ribosomal protein S27a (GenBank Accession No. 354483686), kallikrein-11 (GenBank Accession No. 625217455), serine protease HTRA1 isoform X1 (GenBank Accession No. 625222219), complement C1r subcomponent (GenBank Accession No. 625183025), actin, aortic smooth muscle isoform X1 (GenBank Accession No. 625206860), heat shock cognate 71 kDa protein (GenBank Accession No. 350539823), peroxiredoxin-1 (GenBank Accession No. 350537945), polyubiquitin (GenBank Accession No. 346986309), glutathione S-transferase Y1 (GenBank Accession No. 354505868), 40S ribosomal protein S28 (GenBank Accession No. 625218224), thioredoxin isoform X1 (GenBank Accession No. 625209431), basement membrane-specific heparin sulfate proteoglycan core protein isoform X1 (GenBank Accession No. 625201352), tubulointerstitial nephritis antigen-like protein (GenBank Accession No. 625188472), actin, partial cytoplasmic 2 isoform X2 (GenBank Accession No. 354497282), galectin-1 (GenBank Accession No. 354496408), cornifin alpha (GenBank Accession No. 354504887). In some embodiments of the disclosed methods, the content of HCPs that is reduced in the antibody preparations is a content of HCPs selected from S100-A6, protein S100-A11, phospholipase B-like 2 protein, lysosomal protective protein, ubiquitin-40S ribosomal protein S27a, kallikrein-11, serine protease HTRA1 isoform X1, complement C1r subcomponent, actin, aortic smooth muscle isoform X1, heat shock cognate 71 kDa protein, and peroxiredoxin-1, and combinations thereof. The disclosed methods may be utilized to prepare antibody compositions having a content of one or more of S100-A6, protein S100-A11, phospholipase B-like 2 protein, lysosomal protective protein, ubiquitin-40S ribosomal protein S27a, kallikrein-11, serine protease HTRA1 isoform X1, complement C1r subcomponent, actin, aortic smooth muscle isoform X1, heat shock cognate 71 kDa protein, and peroxiredoxin-1 that is less than about 100 ppm, 50 ppm, 20 ppm, 10 ppm, 5 ppm, 2 ppm, and 1 ppm.

It is particularly desirable to remove those HCPs with an EpiMatrix score of +20 such as Phospholipase B-like 2 protein (PLBL2) (GenBank Accession No. 354497505), S100-A6 (GenBank Accession No. 354478978), protein S100-A11 (GenBank Accession No. 354490016), lysosomal protective protein (GenBank Accession No. 354476738) because of the elevated immunogenicity risk. Other HCPs such as periredoxin-1 are quite persistent and difficult to remove because of their tendency to co-elute with a protein or antibody of interest.

The term “weak acid” refers to an acid with a lowest pKa of >˜4. Examples of weak acids include but are not limited to, acetic acid, succinic acid, and 2-(N-morpholino)ethanesulfonic acid.

The term “strong acid” refers to an acid with a lowest pKa of <˜4. Examples of strong acids include but are not limited to, phosphoric acid, lactic acid, formic acid, malic acid, malonic acid, glycolic acid, citric acid, tartaric acid, and hydrochloric acid.

The term “valency” refers to the combining capacity of an atom. The number of bonds that an atom can form as part of a compound is expressed by the valency of the element. The term “monovalent” refers to an atom, ion, or chemical group with a valence of one, which thus can form one covalent bond.

The term “depth filter” refers to a filter element that uses a porous filtration medium which retains particles throughout the medium (within and on the medium) rather than just on the surface of the medium. Depth filters may additionally have adsorptive capabilities resulting from the chemical properties of the materials from which they are composed. Examples of commercially available depth filters include, but are not limited to a B1HC filter, a X0SP filter, a C0SP filter, a X0HC filter, an Emphaze™ AEX Hybrid Purifier, a Zeta Plus (60ZB05A) filter, a Zeta Plus (90ZB05A) filter, and a Zeta Plus (90ZB08A) filter. The depth filter may be a fully synthetic depth filter comprising a fully synthetic filter media. The depth filter may have a pore size from about 9 microns to about 0.1 microns, from about 2 microns to about 0.1 microns, or about 0.1 microns. The term “depth filtration” refers to the act of passing a liquid material which may be heterogeneous or homogeneous through a depth filter.

The term “ionic strength,” when referring to a solution, is a measure of concentration of ions in that solution. Ionic strength (1) is a function of species concentration, cᵢ, and net charge, zᵢ, for all species. To determine ionic strength, Formula I is used.

$$I = \frac{1}{2} {\sum }_{i} {c}_{i} {z}_{i}^{2} \qquad ( 1 )$$

An “antibody preparation” is the material or solution provided for a purification process or method which contains a therapeutic or diagnostic antibody or antigen-binding fragment thereof of interest and which may also contain various impurities. Non-limiting examples may include, for example, harvested cell culture fluid (HCCF), harvested cell culture material, clarified cell culture fluid, clarified cell culture material, the capture pool, the recovered pool, and/or the collected pool containing the therapeutic or diagnostic antibody of interest after one or more centrifugation steps, and/or filtration steps, the capture pool, the recovered pool and/or the collected pool containing the therapeutic or diagnostic antibody of interest after one or more purification steps.

The term “impurities” refers to materials that are different from the desired anti-N3pG antibody product. The impurity includes, without limitation: host cell materials, such as host cell proteins, CHOP; leached Protein A; nucleic acid; a variant, size variant, fragment, aggregate or derivative of the desired antibody; endotoxin; viral contaminant; cell culture media component, etc.

The terms “protein” and “polypeptide” are used interchangeably herein to refer to a polymer of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, proteins containing one or more analogs of an amino acid (including, for example, unnatural amino acids, etc.), as well as other modifications known in the art. Examples of proteins include, but are not limited to, antibodies, peptides, enzymes, receptors, hormones, regulatory factors, antigens, binding agents, cytokines, Fc fusion proteins, immunoadhesin molecules, etc.

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (IgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)2, Fv fragments, scFv antibody fragments, scFab, disulfide-linked Fvs (sdFv), a Fd fragment.

The disclosed methods may be performed in order to prepare a drug substance preparation.

The disclosed methods and compositions may utilize or comprise antibodies against Np3Glu Amyloid beta peptide (“anti-Np3G antibodies”). The anti-Np3G antibodies may be used in treating diseases related to Amyloid Beta (A3) peptide aggregation. The cleavage of the amyloid precursor protein (APP) results in Aβ peptides ranging in size from 38 to 43 amino acids. Conversion of Aβ from soluble to insoluble forms having high 3-sheet content and the deposition of these insoluble forms as neuritic and cerebrovascular plaques in the brain has been associated with a number of conditions and diseases, including Alzheimer's disease (AD), Down's syndrome, and cerebral amyloid angiopathy (CAA). The deposits found in plaques are comprised of a heterogeneous mixture of Aβ peptides. N3pGlu A3, also referred to as N3pE, pE3-X, or Aβ_{p3-X}, is an N-terminal truncated form of Aβ peptide and is primarily found in plaque. N3pGlu Aβ lacks the first two amino acid residues at the N-terminus of human Aβ and has a pyroglutamate which was derived from the glutamic acid at the third amino acid position. Although N3pGlu Aβ peptide is a minor component of the deposited Aβ in the brain, studies have demonstrated that N3pGlu Aβ peptide has aggressive aggregation properties and accumulates early in the deposition cascade. Antibodies to N3pGlu Aβ are known in the art. For example, U.S. Pat. No. 8,679,498 discloses human N3pGlu Aβ antibodies (e.g. B12L; also known as LY3002813) and methods of treating diseases, such as Alzheimer's disease, with said antibodies. U.S. Pat. No. 10,647,759 discloses N3pG Ab antibodies including “Antibody 201c” and methods of treating diseases, such as Alzheimer's disease, with said antibodies. The anti-Np3Glu antibodies of the disclosed methods and compositions may specifically bind to an epitope present within Ab which is Pyr-EFRHDSGYEVHHQK (i.e., pE3-16).

The disclosed methods and compositions may utilize or comprise antibodies against the spike protein of sudden acute respiratory syndrome coronavirus 2 (SARS-CoV-2). The term “anti-SARS-CoV2 antibody” as used herein refers to an antibody that binds the spike (S) protein of SARS-CoV-2. The amino acid sequence of SARS-CoV-2 spike (S) protein has been described before, for example, GenBank Accession No: YP_009724390.1.

The term “ultrafiltration” or “filtration” is a form of membrane filtration in which hydrostatic pressure forces a liquid against a semipermeable membrane. Suspended solids and solutes of high molecular weight are retained, while water and low molecular weight solutes pass through the membrane. In some examples, ultrafiltration membranes have pore sizes in the range of 1 m to 100 m. The terms “ultrafiltration membrane” “ultrafiltration filter” “filtration membrane” and “filtration filter” may be used interchangeably. Examples of filtration membranes include but are not limited to polyvinylidene difluoride (PVDF) membrane, cellulose acetate, cellulose nitrate, polytetrafluoroethylene (PTFE, Teflon), polyvinyl chloride, polyethersulfone, glass fiber, or other filter materials suitable for use in a cGMP manufacturing environment.

As used herein, numeric ranges are inclusive of the numbers defining the range.

The term “EU numbering”, which is recognized in the art, refers to a system of numbering amino acid residues of immunoglobulin molecules. EU numbering is described, for example, at Kabat et al., Sequences of Proteins of Immunological Interest, 5th Ed. Public Health Service, National Institutes of Health, Bethesda, M D. (1991); Edelman, G. M, et al., Proc. Natl. Acad. USA, 63, 78-85 (1969); and http://www.imgt.org/IMGTScientificChart/Numbering/Hu_IGHGnber.html#refs. The term “Kabat numbering” is recognized in the art as referring to a system of numbering amino acid residues which are more variable (i.e., hypervariable) than other amino acid residues in heavy and light chain variable regions (see, for example, Kabat, et al., Ann. NY Acad. Sci. 190:382-93 (1971); Kabat et al., Sequences of Proteins of Immunological Interest, Fifth Edition, U.S. Department of Health and Human Services, NIH Publication No. 91-3242 (1991)). The term “North numbering”, refers to a system of numbering amino acid residues which are more variable (i.e., hypervariable) than other amino acid residues in heavy and light chain variable regions and is based, at least in part, on affinity propagation clustering with a large number of crystal structures, as described in (North et al., A New Clustering of Antibody CDR Loop Conformations, Journal of Molecular Biology, 406:228-256 (2011).

As used herein, the term “affinity chromatography” refers to a chromatographic method for separating biochemical mixtures (e.g., a protein and undesired biomolecule species) based on specific, reversible interactions between biomolecules. Exemplary embodiments of affinity chromatography include Protein A affinity, Protein G affinity, protein L affinity, kappa affinity ligand chromatography (such as CaptureSelect™ KappaXL™, KappaSelect™, KappaXP™) or lambda affinity ligand chromatography.

A protein of the present disclosure can be incorporated into a pharmaceutical composition which can be prepared by methods well known in the art and which comprise a protein of the present disclosure and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with the protein, retains the molecule's activity and is non-reactive with the patient's immune system.

Expression vectors capable of directing expression of genes to which they are operably linked are well known in the art. Expression vectors can encode a signal peptide that facilitates secretion of the polypeptide(s) from a host cell. The signal peptide can be an immunoglobulin signal peptide or a heterologous signal peptide. Each of the expressed polypeptides may be expressed independently from different promoters to which they are operably linked in one vector or, alternatively, may be expressed independently from different promoters to which they are operably linked in multiple vectors. The expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

A host cell refers to cells stably or transiently transfected, transformed, transduced or infected with one or more expression vectors expressing one or more protein of the present disclosure. Creation and isolation of host cell lines producing proteins of the present disclosure can be accomplished using standard techniques known in the art. Mammalian cells are preferred host cells for expression of proteins of the present disclosure. Particular mammalian cells include HEK 293, NSO, DG-44, and CHO. Preferably, the proteins are secreted into the medium in which the host cells are cultured, from which the proteins can be recovered or purified by for example using conventional techniques. For example, the medium may be applied to and eluted from a Protein A affinity chromatography column and/or a kappa affinity ligand or lambda affinity ligand chromatography column. Undesired biomolecule species including soluble aggregate and multimers may be effectively removed by common techniques, including size exclusion, hydrophobic interaction, ion exchange, or hydroxyapatite chromatography. The product may be immediately frozen, for example at −70° C., refrigerated, or may be lyophilized. Various methods of protein purification may be employed, and such methods are known in the art and described, for example, in Deutscher, Methods in Enzymology 182: 83-89 (1990) and Scopes, Protein Purification: Principles and Practice, 3rd Edition, Springer, NY (1994).

Also disclosed herein are pharmaceutical compositions comprising an antibody or an antigen-binding fragment thereof, wherein the antibody or antigen-binding fragment thereof was prepared by a process comprising purifying the antibody from a mammalian host cell. In the disclosed pharmaceutical compositions comprising an antibody, the total content of host cell proteins (HCPs) in the composition typically is less than about 100 ppm, 50 ppm, 20 ppm, 10 ppm, 5 ppm, or 1 ppm (e.g., as measured by LCMS). In some embodiments of the disclosed pharmaceutical compositions, the antibody of the disclosed pharmaceutical compositions binds to human N3pGlu Aβ (anti-N3pGlu Aβ antibody). In some embodiments, the mammalian cell is a Chinese hamster ovary (CHO) cell.

The disclosed pharmaceutical compositions typically comprise an antibody or an antigen-binding fragment thereof, which may be an anti-N3pGlu Aβ antibody. In some embodiments, the antibody is a monoclonal antibody, a chimeric antibody, a humanized antibody, a human antibody, a bispecific antibody, or an antibody fragment. In some embodiments, the antibody is an IgG1 antibody.

The disclosed pharmaceutical compositions may comprise an anti-N3pGlu Aβ antibody. In some embodiments, the anti-N3pGlu Aβ antibody comprises a heavy chain (HC) and a light chain (LC), wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein

LCDR1 is
(SEQ ID NO: 17)
KSSQSLLYSRGKTYLN,
LCDR2 is
(SEQ ID NO: 18)
AVSKLDS,
LCDR3 is
(SEQ ID NO: 19)
VQGTHYPFT,
HCDR1 is
(SEQ ID NO: 20)
GYDFTRYYIN,
HCDR2 is
(SEQ ID NO: 21)
WINPGSGNTKYNEKFKG,
and
HCDR3 is
(SEQ ID NO: 22)
EGITVY.

In some embodiments of the disclosed pharmaceutical compositions, the compositions comprise an anti-N3pGlu Aβ antibody, wherein the antibody comprises a LCVR and a HCVR, wherein the LCVR is

(SEQ ID NO: 13)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIK

### and the HCVR is

(SEQ ID NO: 14)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSS.

In some embodiments of the disclosed pharmaceutical compositions, the compositions comprise an anti-N3pGlu Aβ antibody, wherein the LC of the anti-N3pGlu Aβ antibody is

(SEQ ID NO: 15)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

and the HC of the anti-N3pGlu Aβ antibody is

(SEQ ID NO: 16)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPG.

In some embodiments of the disclosed compositions, the compositions comprise donanemab.

In some embodiments, the disclosed compositions comprise an anti-N3pGlu Aβ antibody that comprises a heavy chain (HC) and a light chain (LC), wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is RASQSLGNWLA (SEQ ID NO: 27), LCDR2 is YQASTLES (SEQ ID NO: 28). LCDR3 is QHYKGSFWT (SEQ ID NO: 29), HCDR1 is AASGFTFSSYPMS (SEQ ID NO: 30), HCDR2 is

(SEQ ID NO: 31)
AISGSGGSTYYADSVKG,
and
HCDR3 is
(SEQ ID NO: 32)
AREGGSGSYYNGFDY.

In some embodiments of the disclosed pharmaceutical compositions, the compositions comprise an anti-N3pGlu Aβ antibody, wherein the antibody comprises a LCVR and a HCVR, wherein the LCVR is

(SEQ ID NO: 23)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIK

### and the HCVR is

(SEQ ID NO: 24)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSS.

In some embodiments of the disclosed pharmaceutical compositions, the compositions comprise an anti-N3pGlu Aβ antibody, wherein the LC of the anti-N3pGlu Aβ antibody is

(SEQ ID NO: 25)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

and the HC of the anti-N3pGlu Aβ antibody is

(SEQ ID NO: 26)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPG.

In some embodiments of the disclosed compositions, the compositions comprise Antibody 201c as referenced in U.S. Pat. No. 10,647,759.

In the disclosed pharmaceutical compositions comprising an anti-N3pG antibody, which may include an anti-N3pGlu antibody such as donanemab, the pharmaceutical compositions may have a reduced total content of host cell proteins (HCPs). In some embodiments, the compositions comprise less than about 100 ppm, 50 ppm, 20 ppm, 10 ppm, 5 ppm, or 1 ppm of HCPs (e.g., as measured by LCMS). In some embodiments, the compositions comprise less than about 100 ppm, 50 ppm, 20 ppm, 10 ppm, 5 ppm, or 1 ppm of HCPs selected from the following HCPs and combinations thereof: protein S100-A6, protein S100-A11, phospholipase B-like 2 protein, lysosomal protective protein, ubiquitin-40S ribosomal protein S27a, kallikrein-11, serine protease HTRA1 isoform X1, complement C1r subcomponent, actin, aortic smooth muscle isoform X1, heat shock cognate 71 kDa protein, peroxiredoxin-1.

In the disclosed pharmaceutical compositions comprising an anti-N3pG antibody, the compositions may comprise less than about 100 ppm, 50 ppm, 20 ppm, 10 ppm, 5 ppm, or 1 ppm of protein S100-A6 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of protein S100-A11 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of phospholipase B-like 2 protein (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of lysosomal protective protein (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of ubiquitin-40S ribosomal protein S27a (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of kallikrein-11 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm serine protease HTRA1 isoform X1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm complement C1r subcomponent (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm actin, aortic smooth muscle isoform X1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm actin, aortic smooth muscle isoform X1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm heat shock cognate 71 kDa protein (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm peroxiredoxin-1 (e.g., as measured by LCMS).

In the disclosed pharmaceutical compositions comprising an antibody, which may include an anti-N3pGlu antibody such as Antibody 201c, the pharmaceutical compositions may have a reduced total content of host cell proteins (HCPs). In some embodiments, the compositions comprise less than about 100 ppm, 50 ppm, 20 ppm, 10 ppm, 5 ppm, or 1 ppm of HCPs (e.g., as measured by LCMS). In some embodiments, the compositions comprise less than about 100 ppm, 50 ppm, 20 ppm, 10 ppm, 5 ppm, or 1 ppm of HCPs selected from the following HCPs and combinations thereof: polyubiquitin, lysosomal protective protein, glutathione S-transferase Y1, 40S ribosomal protein S28, thioredoxin isoform X1, basement membrane-specific heparan sulfate proteoglycan core protein isoform X1, tubulointerstitial nephritis antigen-like protein, actin-partial cytoplasmic 2 isoform X2, galectin-1, peroxiredoxin-1, and cornifin alpha.

In the disclosed pharmaceutical compositions comprising an anti-N3pG antibody, the compositions may comprise less than about 100 ppm, 50 ppm, 20 ppm, 10 ppm, 5 ppm, or 1 ppm of polyubiquitin (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of lysosomal protective protein (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of glutathione S-transferase Y1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of glutathione S-transferase Y1 e.g., (as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of 40S ribosomal protein S28 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of thioredoxin isoform X1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of basement membrane-specific heparan sulfate proteoglycan core protein isoform X1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of tubulointerstitial nephritis antigen-like protein (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of actin-partial cytoplasmic 2 isoform X2 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of galectin-1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of peroxiredoxin-1 (e.g., as measured by LCMS). In the disclosed pharmaceutical compositions comprising an anti-N3pG antibody, the compositions may comprise less than about 100 ppm, 50 ppm, 20 ppm, 10 ppm, 5 ppm, or 1 ppm of cornifin alpha (e.g., as measured by LCMS).

### EXAMPLES

Host cell protein (HCP) measurements by LCMS: to assess purification impact on host cell protein (HCP) levels in the examples which follow, samples are analyzed by peptide mapping/LC-MS/MS HCP profiling via, e.g., a Ultra Performance Liquid Chromatography (UPLC) coupled to a Thermo Scientific mass spectrometer. Methods for detecting HCPs have been disclosed in the art. (See, e.g., Huang et al., “A Novel Sample Preparation for Shotgun Proteomics Characterization of HCPs in Antibodies,” Anal. Chem. 2017, 89, 5436-5444.) In this analysis, the samples are subjected to digestion by trypsin, reduced/precipitated with dithiothreitol (DTT), followed by transfer and acidification of the supernatant in a HPLC vial for LC-MS/MS analysis. The LC-MS/MS data is analyzed by Proteome Discoverer against CHO-K1 protein database with added antibody, spike, and control protein sequences. The HCP concentration is reported as total parts per million (ppm) of HCP per sample for total HCP content (e.g., ng of HCP per mg of product). Additionally, the concentrations of certain HCPs, (e.g., phospholipase B-like 2 protein (PLBL2) and lysosomal protective protein) are also provided.
HCP measurements by ELISA: HCP levels concentration in the samples are also assessed in the examples which follow by an ELISA assay using a Gyrolab© CHO-HCP Kit 1 (Cygnus Technologies, performed per manufacturer instructions). The HCP results concentration are reported as total parts per million (ppm) of HCP per sample for total HCP content.

### Example 1—HCP Reduction in mAb1 (Etesevimab) Purification Process

Protein Capture step: A sanitized Protein A column (MabSelect SuRe Protein A media) is equilibrated and mAb1 (etesevimab) cell-free bioreactor harvest is loaded onto the Protein A column and three washes of the Protein A column are performed using 20 mM Tris (pH 7.0) as the last wash. mAb1 is eluted from the column using 5 column volumes (CVs) of 20 mM acetic acid+5 mM phosphoric acid. The main product fraction is collected into a single bulk fraction by using absorbance-based peak cutting on the frontside and backside.
Low pH Viral Inactivation Step and Neutralization Step: The pH of the main product fraction (protein capture eluate bulk fraction) containing mAb1 is adjusted to a pH between 3.30 and 3.60 by the addition of 20 mM HCl for low pH viral inactivation. The mixture is incubated at 18° C. to 25° C. for 180 min. The mixture is then neutralized to a pH of 7.0 using 250 mM Tris base pH unadjusted buffer.
Depth Filtration Step: A depth filter (X0SP, Millipore) is flushed with water for injection (WFI). The mAb1 mixture, obtained from the low pH viral inactivation step and neutralization step, is applied to the depth filter with a loading of 1200 g/m² (grams of mAb per m² of depth filter membrane area). The loaded depth filter is flushed with WFI. The filtrate from the depth filter, optionally inclusive of the post-loading WFI flush, is neutralized to pH 8.0 using 250 mM Tris base pH unadjusted buffer.
Anion Exchange (AEX) Chromatography Step: A sanitized column (Q Sepharose Fast Flow Anion Exchange Chromatography Media, or QFF) is equilibrated with 2 CVs of 20 mM Tris (pH 8.0). The mAb1 solution, obtained from the depth filtration step, is loaded onto the column at a loading of 25 to 100 g per liter of resin, and an additional wash is performed with the equilibration buffer. mAb1 is collected by absorbance-based peak cutting on the frontside and backside of the peak area formed by the unbound fraction plus the additional wash.
Results: Using the purification process described, the total HCP level as measured by LC-MS is:
23299 ppm after Protein A elution;
13 ppm after X0SP depth filtration;
2 ppm after AEX chromatography.
   Depth filter Set 1 assessment for mAb1: mAb1 is processed through Protein A, low pH viral inactivation, neutralization, and depth filtration steps essentially as described above. Four different depth filters: Emphaze™ AEX Hybrid Purifier, Zeta Plus BC25-60ZB05A, Zeta Plus BC25-90ZB05A, and Zeta Plus BC25-90ZB08A (3M) are tested at a loading of 2000 g/m² as shown in Table 1. The results in Table 1 show a significant reduction in total HCP content after depth filtration by LCMS and/or ELISA for the 4 depth filters tested when compared to the total HCP content observed after Protein A elution.

TABLE 1
mAb1 total HCP content before and after depth filtration

| Total HCP content | | | Total HCP content | |
|---|---|---|---|---|
| after Protein A | | | after depth | |
| elution (ppm) | | | filtration (ppm) | |
| LCMS | ELISA | Depth filter | LCMS | ELISA |
| 28901 | 527 | Emphaze ™ AEX | not available | 16 |
| | | Hybrid Purifier | | |
| | | Zeta Plus BC25 - | 31 | 8 |
| | | (60ZB05A) | | |
| | | Zeta Plus BC25 - | 29 | 7 |
| | | (90ZB05A) | | |
| | | Zeta Plus BC25 - | 24 | 6 |
| | | (90ZB08A) | | |

### Example 2—HCP Reduction in mAb2 (Bamlanivimab) Purification Process

Protein A elution buffer comparison: mAb2 is prepared essentially as described for mAb1 in Example 1 with the following exceptions: 1) after low pH viral inactivation and before depth filtration, the solution is neutralized to a pH of 7.25 instead of 7.0 using 250 mM Tris base pH unadjusted buffer, 2) mAb 2 is eluted from the Protein A capture column using the buffer combinations listed in Table 2, and 3) the AEX chromatography is performed using Poros XQ resin. HCP content (both total HCP levels and PLBL2 levels) is assessed via LCMS, after purification unit operations as listed in Tables 2 and 3. The results in Tables 2 and 3, show that total HCP and PLBL2 content is reduced for all 3 buffer combinations tested, after the depth filtration step. Specifically, the 20 mM acetic acid+5 mM phosphoric acid and 20 mM acetic acid+5 mM L-lactic acid showed a greater reduction of total HCP and PLBL2 of less than 20 ppm when compared to the 20 mM acetic acid+5 mM citric acid combination after depth filtration.

TABLE 2
mAb2 total HCP content using different Protein A elution buffers

| | | | Total HCP by |
|---|---|---|---|
| | Total HCP by | Total HCP by | LCMS detection |
| Protein A | LCMS detection | LCMS detection | after AEX |
| elution | after Protein A | after X0SP depth | chromatography |
| buffer | elution (ppm) | filtration (ppm) | (ppm) |
| 20 mM acetic | 71022 | 469 | 55 |
| acid + 5 mM | | | |
| citric acid | | | |
| 20 mM acetic | 77892 | 7 | 11 |
| acid + 5 mM | | | |
| phosphoric acid | | | |
| 20 mM acetic | 78669 | 16 | Below limit of |
| acid + 5 mM | | | quantitation |
| L-lactic acid | | | |

TABLE 3
mAb2 PLBL2 content using different Protein A elution buffers

| | | | PLBL2 by LCMS |
|---|---|---|---|
| | PLBL2 by LCMS | PLBL2 by LCMS | detection after |
| Protein A | detection after | detection after | AEX |
| elution | Protein A elution | X0SP depth | chromatography |
| buffer | (ppm) | filtration (ppm) | (ppm) |
| 20 mM acetic | 356 | 454 | 8 |
| acid + 5 mM | | | |
| citric acid | | | |
| 20 mM acetic | 351 | Below limit of | Below limit of |
| acid + 5 mM | | quantitation | quantitation |
| phosphoric acid | | | |
| 20 mM acetic | 404 | Below limit of | Below limit of |
| acid + 5 mM | | quantitation | quantitation |
| L-lactic acid | | | |

Depth filter set 2 assessment: mAb 2 is prepared essentially as described for mAb1 with the following exceptions: 1) after low pH viral inactivation and before depth filtration, neutralize the pH of the solution to a pH of 7.25 instead of 7.0 using 250 mM Tris base pH unadjusted buffer, and 2) depth filtration is performed with the depth filters shown in Table 4. Table 4 shows total HCP and PLBL2 content after depth filtration using various depth filters at a loading of 1500 g/m². All 3 set 2 depth filters tested (X0SP, C0SP, X0HC, (Millipore)) show significant reduction in total HCP and PLBL2 content of less than 20 ppm after depth filtration.

TABLE 4
mAb2 HCP total and PLBL2 content
before and after depth filtration

| | | | Total HCP | PLBL2 |
|---|---|---|---|---|
| Total HCP | PLBL2 | | content by | content by |
| content by | content by | | LCMS | LCMS |
| LCMS after | LCMS after | | after depth | after depth |
| Protein A | Protein A | Depth | filtration | filtration |
| elution (ppm) | elution (ppm) | filter | (ppm) | (ppm) |
| 74528 | 543 | X0SP | 3 | Below |
| | | | | limit of |
| | | | | quantitation |
| | | C0SP | 18 | 5 |
| | | X0HC | 2 | Below |
| | | | | limit of |
| | | | | quantitation |

### Example 3. HCP Reduction in mAb3 (Bebtelovimab) Purification Process

mAb3 is prepared using the protein capture, low pH viral inactivation, neutralization, and depth filtration steps essentially as described for mAb1 in Example 1, except using a X0SP depth filter with a loading of 900 g/m². Using the described purification process the total HCP level as measured by LCMS is:
179964 ppm after the Protein A elution,
77 ppm after X0SP (Millipore) depth filtration.

### Example 4. HCP Reduction in Bispecific Antibody (mAb4) Purification Process

A bispecific antibody mAb4 is prepared using the protein capture step essentially as described for mAb1 in Example 1, except using a Protein L affinity capture column (Cytiva) and eluting with the buffer systems shown in Table 5. The total HCP content is measured by ELISA giving a range of about 1300 to about 2500 ppm. Following protein capture, low pH viral inactivation is performed essentially as described for mAb1 in Example 1, except using the titrants listed in Table 5, followed by neutralization up to pH 7.0 using 500 mM Tris base pH unadjusted buffer. Then, the depth filtration step is performed as described for mAb1 in Example 1 using a X0SP depth filter at a loading of 1200 g/m². The HCP content is measured after depth filtration by ELISA.

The results in Table 5, show significant reduction in total HCP content to less than ≤50 ppm for Entries 1 to 7 following depth filtration, where the ionic strength of the mixtures applied to the depth filter was less than 45 mM. In addition, a correlation between the ionic strength of the mixtures applied to the depth filter and the total HCP content after the depth filtration. Furthermore, Entry 2 shows that ionic strength can be decreased by diluting the buffer, providing low HCP content after depth filtration, however the volume increase from dilution can be disadvantageous to manufacturing processes.

TABLE 5
HCP levels in mAb4 preparations following
Protein L elution and depth filtration

| | | | Ionic strength | Total HCP |
|---|---|---|---|---|
| | | | of mixture | content by |
| | | Low pH viral | applied to | ELISA after |
| | Protein L | inactivation | depth filter | X0SP depth |
| Entry | elution buffer | titrant | (mM) | filtration (ppm) |
| 1 | 20 mM acetic | 20 mM acetic | 38 | 38 |
| | acid + 10 mM | acid + 10 mM | | |
| | phosphoric acid | phosphoric acid | | |
| 2 | 20 mM acetic | 20 mM acetic | 13 (after 1:2 | 18 |
| | acid + 10 mM | acid + 10 mM | H₂O dilution)* | |
| | phosphoric acid | phosphoric acid | | |
| 3 | 20 mM acetic | 20 mM HCl | 36 | 35 |
| | acid + 10 mM | | | |
| | phosphoric acid | | | |
| 4 | 20 mM acetic | 20 mM HCl | 27 | 30 |
| | acid + 5 mM | | | |
| | phosphoric acid | | | |
| 5 | 20 mM acetic | 20 mM HCl | 23 | 26 |
| | acid + 5 mM | | | |
| | formic acid | | | |
| 6 | 20 mM acetic | 200 mM | 43 | 50 |
| | acid + 10 mM | phosphoric acid | | |
| | phosphoric acid | | | |
| 7 | 20 mM acetic | 15 mM | 37 | 36 |
| | acid + 10 mM | phosphoric acid | | |
| | phosphoric acid | | | |
| 8 | 20 mM acetic | 1000 mM | 64 | 209 |
| | acid + 10 mM | citric acid | | |
| | phosphoric acid | | | |

*following low pH viral inactivation and neutralization to pH 7.0 with 500 mM Tris, the mAb4 solution is diluted with 2 parts water (1:2 ratio of mAb4 solution:H₂O)

### Example 5. HCP Reduction in mAb5 (Donanemab) Purification Processes

A mAb5 preparation is prepared using the steps as essentially described below: protein capture, low pH viral inactivation and neutralization, depth filtration, anion exchange (AEX) chromatography, cation exchange (CEX) chromatography, viral filtration and tangential flow filtration (TFF).

### Protein Capture Step:

Capture and purify the antibody by reducing process-related impurities such as residual HCPs and residual DNA. A sanitized Protein A column (MabSelect Protein A media) is equilibrated and a monoclonal antibody (mAb5 (donanemab) expressed from CHO cell) cell-free bioreactor harvest is loaded onto the Protein A column and three washes of the Protein A column are performed using 20 mM Tris (pH 7.0) as the last wash. The antibody is eluted from the column using 5 column volumes (CVs) of 20 mM acetic acid+5 mM citric acid. The main product fraction is collected into a single bulk fraction by using absorbance-based peak cutting on the frontside and backside.

### Low pH Viral Inactivation Step and Neutralization Step:

Inactivate low pH susceptible viruses, reduce residual HCP, residual protein A, residual DNA and total aggregates. Viral inactivation is conducted by adjusting the pH of the collected main product fraction (protein capture eluate bulk fraction) containing the mAb to a pH between 3.30 and 3.60 by the addition of 20 mM acetic acid, 5 mM citric acid. The mixture is incubated at 18° C. to 25° C. for about 180 min. The mixture is then neutralized to a pH of 5 to 7.0, preferably pH 5.0, using 250 mM Tris base pH unadjusted buffer.

### Depth Filtration Step:

A separate depth filter (B1HC, Millipore) is flushed with water for injection (WFI) for each test condition (pH 5 with B1HC). The mAb mixture, obtained from the low pH viral inactivation step and neutralization step, is applied to the depth filter with a target loading of approximately 500-1500 g/m² (grams of mAb per m² of depth filter membrane area). The loaded depth filter is flushed with WFI. The filtrate from the depth filter, optionally inclusive of the post-loading WFI flush, is neutralized to pH 7.25 using 250 mM Tris base pH unadjusted buffer. A calculated volume of 20 mM Tris, 1 M NaCl, pH 7.0 buffer to added to a final NaCl concentration of 50 mM.

### Anion Exchange (AEX) Chromatography Step:

Reduce potential viral contaminants. A sanitized Poros XQ (or Sartobind Q or Poros HQ) anion exchange (AEX) column (is pre-equilibrated with 2 CV of 20 mM Tris, 1 M NaCl, pH 7.0 buffer followed by 3 CVs of equilibration buffer 20 mM Tris 50 mM NaCl, (pH 7.25). The mAb solutions from each of the of depth filter conditions were flowed through the AEX column in discrete runs based upon depth filter condition, obtained from the depth filtration step, is loaded onto the column at a loading of approximately 100 g-200 g per liter of resin (e.g., approximately 150 g per liter of resin), and an additional wash is performed with the equilibration buffer. mAb is collected from the start of loading until the end of wash.

### Cation Exchange (CEX) Chromatography Step:

Reduce total aggregates, reduce residual HCP and reduce residual protein A. The different AEX intermediates were pH adjusted from approximately 7.25 to 5.0 with the addition of 0.1 N acetic acid before loading onto the equilibrated (20% Mobile Phase B or equivalent to 20 mM sodium acetate, 200 mM sodium chloride, pH 5.0) CEX chromatography resin (POROS™ HS or UNOsphere S). The AEX process intermediate at pH 5.0 is blended with 15% Mobile Phase B (corresponding to 193 mM sodium chloride) at the point of loading onto the CEX column. Column load was approximately 25 grams of mAb per liter of resin. After loading, the column is washed with 20% Mobile Phase B (equivalent to 20 mM sodium acetate, 200 mM sodium chloride, pH 5.0) to facilitate removal of unbound impurities. mAb is then eluted from the column with a linear gradient from 20%-55% Mobile Phase B over 10 column volumes (200 to 550 mM sodium chloride gradient in a 20 mM sodium acetate, pH 5.0 buffer). To ensure complete elution of product, the linear gradient may be followed by an isocratic hold at 55% Mobile Phase B (equivalent to 20 mM sodium acetate, 550 mM sodium chloride, pH 5.0). During elution, a UV-based cut on the front-side at NLT 4.8 AU/cm initiates CEX eluate collection and continues through the peak apex until the back-side cut is made at NLT 2.4 AU/cm. The column is regenerated and sanitized with a 1 N sodium hydroxide solution. The column may be stored in 0.01 N sodium hydroxide. The preparations then are analyzed for HCP content using LCMS.

### Viral Filtration:

Remove potential viral contaminants. Viral filtration is performed through a Viresolve Pro, Planova 20N or Planova BioEX membrane.

### Tangential Flow Filtration (TFF):

Exchange the viral filtrate process intermediate into the appropriate matrix for final drug substance (DS) preparation and concentrate the antibody to the appropriate range for final DS preparation. TFF is performed on a 30 kDa PES or 30 kDa Regenerated Cellulose membrane.

### Drug Substance Dispensing:

After TFF, a surfactant is added to complete the drug substance formulation and dispensed into an approved container closure system for storage and transport at the appropriate temperature prior to drug product manufacture.

### Measurement of HCP Content by LC-MS

HCP content was measured by LC-MS as described below. For mAb5 Batch 1 and mAb5 Batch 2, HCP content was measured after the Protein Capture Step, after low pH viral inactivation, after AEX and after CEX. For mAb5 Batches 3-5, HCP content was measured prior to drug substance dispensing. The results are shown in Tables 6a and 6b and Table 7 below.

### Sample Preparation

The aliquot containing ˜1 mg protein of each sample or control was added to pure water to 193 mL. The solution was mixed with 5.0 mL of 1 M tris-HCl buffer, pH 8, 1.0 mL aliquot of four protein mixture and then treated with 1 mL of 2.5 mg/mL r-trypsin at 37° C. for overnight. Each digest was mixed with 2.0 mL of 50 mg/mL DTT solution and the heat at 90° C. for 15 minutes. The precipitate was observed. Vortexed the samples vigorously for 2×30s. Each sample was centrifuged at 13200 rpm for 3 minutes; 120 mL of the supernatant was transferred into HPLC vial. The samples in the HPLC vials were then mixed with 5.0 μL of 20% TFA in H₂O for LC/MS analysis.

### LC/MS/MS Method

The prepared tryptic peptides were analyzed using UPLC-MS/MS. Samples were directly injected onto a Waters Acquity UPLC CSH C18 (Milford, MA, U.S.A.) (2.1×50 mm, 1.7 m particle size) at a volume of 50 μL. The column was heated to 60° C. during analysis. Separation was performed on a Waters Acquity UPLC system with mobile phase A consisting of 0.1% formic acid in water and mobile phase B consisting of 0.1% formic acid in acetonitrile with equilibrating at 0% mobile phase B for 2 min at 200 L/min, linearly increasing from 0% to 10% over 23 min, to 20% B over 57 min, to 30% over 30 min at a flow rate of 50 L/min, followed with multiple zigzag wash cycles at a flow rate of 400 L/min. Mass spectrometric analysis was performed on a Thermo Scientific Q Exactive Plus mass spectrometer (Bremen, Germany). Data-dependent MS/MS was performed as follows: the first event was the survey positive mass scan (m/z range of 230-1500) followed by 10 HCD events (28% NCE) on the 10 most abundant ions from the first event. Ions were generated using a sheath gas flow rate of 15, an auxiliary gas flow rate of 5, a spray voltage of 4 kV, a capillary temperature of 320° C., and an S-Lens RF level of 50. Resolution was set at 35 000 (AGC target of 5E6) and 17 500 (AGC target of 5E4) for survey scans and MS/MS events, respectively. The maximum ion injection time was 250 ms for survey scan, 300 ms for the other scans. The dynamic exclusion duration of 60s was used with a single repeat count.

### HCP Identification and Quantification

A customized protein database composed of sequences obtained from the CHO-K1_refseq_2014 Protein.fasta database (downloaded 08/23/2014 from http://www.chogenome.org) was developed to predict the identities of HCPs from the MS/MS data. The MS/MS data was searched with a mass tolerance of 10 ppm and 0.02 Da, and a strict false discovery rate (FDR)<1% against this database using the Proteome Discoverer software package, version 1.4 or 2.3 (Thermo Scientific, Bremen, Germany) with Sequest HT searching. Further peptide/protein filtering was performed by eliminating proteins that had scored 0 and single spectrum hit, or single spectrum hit and ≥10 ppm and contaminated human proteins. Protein area from the top 3 peptides (if possible) for each HCP and the areas for the three spiked proteins, r-trypsin, PCSK9, and ADH1 were used to calculate individual HCP concentration (ppm or ng HCP/mg mAb).

TABLE 6a
In process LC-MS HCP content for Batch 1 of mAb5

| | | HCP content | HCP content | | |
|---|---|---|---|---|---|
| | | after | after Low | | |
| | | Protein | pH viral | HCP content | HCP content |
| | EpiMatrix | Capture | inactivation | after AEX | after CEX |
| HCP ID | Score | (ppm) | (ppm) | (ppm) | (ppm) |
| Total | N/A | 101023 | 1685 | 943 | 42.2 |
| protein S100- | 52.84 | 6.6 | 5.9 | 3.9 | Below limit |
| A6 | | | | | of |
| | | | | | quantitation |
| protein S100- | 48.79 | 8.8 | 1.8 | Below limit | Below limit |
| A11 | | | | of | of |
| | | | | quantitation | quantitation |
| phospholipase | 32.89 | 547 | 24.4 | 14.3 | 12.2 |
| B-like 2 | | | | | |
| protein | | | | | |
| lysosomal | 29.45 | 227.7 | 102.7 | 26 | Below limit |
| protective | | | | | of |
| protein | | | | | quantitation |
| ubiquitin-40S | 1.9 | 9.9 | 9.2 | 13.4 | Below limit |
| ribosomal | | | | | of |
| protein S27a | | | | | quantitation |
| Kallikrein-11 | −12.83 | Below limit | Below limit | Below limit | Below limit |
| | | of | of | of | of |
| | | quantitation | quantitation | quantitation | quantitation |
| serine | −13 | 1950.6 | 260.3 | 145.6 | Below limit |
| protease | | | | | of |
| HTRA1 | | | | | quantitation |
| isoform X1 | | | | | |
| thioredoxin | −15.94 | 14.5 | 4.8 | 2.1 | 1.5 |
| isoform X1 | | | | | |
| complement | −23.01 | 644.9 | 28.5 | 23.6 | 16.8 |
| C1r | | | | | |
| subcomponent | | | | | |
| actin, aortic | −34.63 | Below limit | Below limit | Below limit | Below limit |
| smooth | | of | of | of | of |
| muscle | | quantitation | quantitation | quantitation | quantitation |
| isoform X1 | | | | | |
| galectin-1 | −45.49 | 36.4 | 2.7 | Below limit | Below limit |
| | | | | of | of |
| | | | | quantitation | quantitation |
| heat shock | −47.2 | 579.3 | 14.8 | 32.4 | Below limit |
| cognate 71 | | | | | of |
| kDa protein | | | | | quantitation |
| peroxiredoxin-1 | −50.43 | 465.4 | 127.6 | 108.3 | 22.6 |
| cornifin alpha | −109.26 | 68.7 | 11.1 | 12.6 | Below limit |
| | | | | | of |
| | | | | | quantitation |

TABLE 6b
In process LC-MS HCP content for Batch 2 of mAb5

| | | HCP content | HCP content | | |
|---|---|---|---|---|---|
| | | after | after Low | | |
| | | Protein | pH viral | HCP content | HCP content |
| | EpiMatrix | Capture | inactivation | after AEX | after CEX |
| HCP ID | Score | (ppm) | (ppm) | (ppm) | (ppm) |
| Total | N/A | 104333 | 1384 | 933 | 70 |
| protein S100- | 52.84 | 63.4 | 5.7 | 5.8 | Below limit |
| A6 | | | | | of |
| | | | | | quantitation |
| protein S100- | 48.79 | 14.3 | 1.8 | Below limit | Below limit |
| A11 | | | | of | of |
| | | | | quantitation | quantitation |
| phospholipase | 32.89 | 507.6 | 19.8 | 12.2 | Below limit |
| B-like 2 | | | | | of |
| protein | | | | | quantitation |
| lysosomal | 29.45 | 229.6 | 75.9 | 19.9 | 12 |
| protective | | | | | |
| protein | | | | | |
| ubiquitin-40S | 1.9 | 8.3 | Below limit | Below limit | Below limit |
| ribosomal | | | of | of | of |
| protein S27a | | | quantitation | quantitation | quantitation |
| Kallikrein-11 | −12.83 | Below limit | Below limit | Below limit | Below limit |
| | | of | of | of | of |
| | | quantitation | quantitation | quantitation | quantitation |
| serine | −13 | 1850.2 | 150.9 | 88.2 | 6.9 |
| protease | | | | | |
| HTRA1 | | | | | |
| isoform X1 | | | | | |
| thioredoxin | −15.94 | 14.6 | 4.2 | 4.2 | 6.5 |
| isoform X1 | | | | | |
| complement | −23.01 | 542.8 | 24.1 | 23.1 | 15.4 |
| C1r | | | | | |
| subcomponent | | | | | |
| actin, aortic | −34.63 | Below limit | Below limit | Below limit | Below limit |
| smooth muscle | | of | of | of | of |
| isoform X1 | | quantitation | quantitation | quantitation | quantitation |
| galectin-1 | −45.49 | 42.7 | 1.7 | Below limit | Below limit |
| | | | | of | of |
| | | | | quantitation | quantitation |
| heat shock | −47.2 | 590.3 | 17 | 49.4 | Below limit |
| cognate 71 | | | | | of |
| kDa protein | | | | | quantitation |
| peroxiredoxin-1 | −50.43 | 499.6 | 101.2 | 108.3 | 27 |
| cornifin alpha | −109.26 | 75 | 12.1 | 11.2 | 1.1 |

TABLE 7
Drug Substance LC-MS HCP content
for Batches 3, 4 and 5 of mAb5

| | | Batch 3 | Batch 4 | Batch 5 |
|---|---|---|---|---|
| | | Drug | Drug | Drug |
| | | Substance | Substance | Substance |
| | EpiMatrix | HCP content | HCP content | HCP content |
| HCP ID | Score | (ppm) | (ppm) | (ppm) |
| Total | N/A | 39.7 | 52.2 | 51.7 |
| protein S100- | 52.84 | 0.4 | 0.4 | 0.4 |
| A6 | | | | |
| protein S100- | 48.79 | 0.3 | 0.4 | 0.6 |
| A11 | | | | |
| phospholipase | 32.89 | 4.3 | 6.2 | 3.5 |
| B-like 2 | | | | |
| protein | | | | |
| lysosomal | 29.45 | 7.1 | 6.8 | 6.1 |
| protective | | | | |
| protein | | | | |
| ubiquitin-40S | 1.9 | 1.1 | 0.6 | 1.3 |
| ribosomal | | | | |
| protein S27a | | | | |
| Kallikrein-11 | −12.83 | 1.0 | 0.0 | 0.0 |
| serine | −13 | 1.8 | 1.6 | 1.7 |
| protease | | | | |
| HTRA1 | | | | |
| isoform X1 | | | | |
| thioredoxin | −15.94 | 0.5 | 0.6 | 0.7 |
| isoform X1 | | | | |
| complement | −23.01 | 5.2 | 4.3 | 5.6 |
| C1r | | | | |
| subcomponent | | | | |
| actin, aortic | −34.63 | 3.2 | 5.1 | 5.0 |
| smooth muscle | | | | |
| isoform X1 | | | | |
| galectin-1 | −45.49 | 0.4 | 5.5 | 3.2 |
| heat shock | −47.2 | 3.2 | 2.7 | 3.8 |
| cognate 71 | | | | |
| kDa protein | | | | |
| peroxiredoxin-1 | −50.43 | 7.9 | 9.9 | 9.1 |
| cornifin alpha | −109.26 | 0.0 | 0.0 | 0.2 |

### Example 6. HCP Reduction in mAb7 (Antibody 201c″ in U.S. Pat. No. 10,647,759) Purification Processes

A mAb7 (Antibody 201c″ in U.S. Pat. No. 10,647,759)(LC is SEQ ID NO: 25; HC is SEQ ID NO: 26) preparation is prepared using the steps as essentially described above in respect of mAb5 with the following minor differences:

### Protein Capture:

Protein A column: MabSelect SuRe
Load: 20-40 g/L
Elution: 20 mM Acetic Acid/5 mM Citric Acid

### Low pH Viral Inactivation and Neutralization:

Titrant: 20 mM Acetic Acid/5 mM Citric Acid, pH 3.45
Time: 180 min
Neutralization: pH 5.0, 500 mM Tris Base

### Aex Chromatography:

Resin: POROS 50 XQ;
Load: 100-200 g/L load
pH: 7.0

### Cex Chromatography:

Resin: POROS 50 HS
Load: 20-40 g/L

HCP content was measured by LC-MS as described in Example 5. For mAb7 Batch 1 and mAb7 Batch 2, HCP content was measured after the Protein Capture Step, after low pH viral inactivation, after AEX, after CEX and after TFF. The results are shown in Tables 8a and 8b

TABLE 8a
In process LC-MS HCP content for Batch 1 of mAb7

| | | HCP | HCP | | | |
|---|---|---|---|---|---|---|
| | | content | content | HCP | HCP | HCP |
| | | after | after Low | content | content | content |
| | | Protein | pH viral | after | after | after |
| | EpiMatrix | Capture | inactivation | AEX | CEX | TFF |
| HCP ID | Score | (ppm) | (ppm) | (ppm) | (ppm) | (ppm) |
| Total | N/A | 14581 | 66.4 | 63.7 | 4 | 1.2 |
| polyubiquitin | 40.81 | 29.2 | 39 | 49 | Below | Below |
| | | | | | limit of | limit of |
| | | | | | quantitation | quantitation |
| lysosomal | 29.45 | 12.5 | Below | Below | Below | Below |
| protective | | | limit of | limit of | limit of | limit of |
| protein | | | quantitation | quantitation | quantitation | quantitation |
| glutathione | 24.04 | Below | Below | Below | Below | Below |
| S- | | limit of | limit of | limit of | limit of | limit of |
| transferase | | quantitation | quantitation | quantitation | quantitation | quantitation |
| Y1 | | | | | | |
| 40S | −9.16 | 1 | Below | Below | Below | Below |
| ribosomal | | | limit of | limit of | limit of | limit of |
| protein S28 | | | quantitation | quantitation | quantitation | quantitation |
| thioredoxin | −15.94 | 4 | Below | Below | 1 | 1 |
| isoform X1 | | | limit of | limit of | | |
| | | | quantitation | quantitation | | |
| basement | −29.68 | 2241 | 11 | Below | Below | Below |
| membrane- | | | | limit of | limit of | limit of |
| specific | | | | quantitation | quantitation | quantitation |
| heparan | | | | | | |
| sulfate | | | | | | |
| proteoglycan | | | | | | |
| core protein | | | | | | |
| isoform X1 | | | | | | |
| tubulointerstitial | −35.46 | 226 | 5 | Below | Below | Below |
| nephritis | | | | limit of | limit of | limit of |
| antigen-like | | | | quantitation | quantitation | quantitation |
| protein | | | | | | |
| actin - | −38.94 | Below | Below | 2 | Below | Below |
| partial | | limit of | limit of | | limit of | limit of |
| cytoplasmic | | quantitation | quantitation | | quantitation | quantitation |
| 2 isoform | | | | | | |
| X2 | | | | | | |
| galectin-1 | −45.49 | 32.6 | 1 | Below | Below | Below |
| | | | | limit of | limit of | limit of |
| | | | | quantitation | quantitation | quantitation |
| peroxiredoxin- | −50.43 | 183.2 | 7 | 10 | 3 | Below |
| 1 | | | | | | limit of |
| | | | | | | quantitation |
| cornifin | −109.26 | 46.8 | 4 | 2 | Below | Below |
| alpha | | | | | limit of | limit of |
| | | | | | quantitation | quantitation |

TABLE 8b
In process LC-MS HCP content for Batch 2 of mAb7

| | | HCP | HCP | | | |
|---|---|---|---|---|---|---|
| | | content | content | HCP | HCP | HCP |
| | | after | after Low | content | content | content |
| | | Protein | pH viral | after | after | after |
| | EpiMatrix | Capture | inactivation | AEX | CEX | TFF |
| HCP ID | Score | (ppm) | (ppm) | (ppm) | (ppm) | (ppm) |
| Total | N/A | 8761 | 70.8 | 106.5 | 7.7 | 0 |
| polyubiquitin | 40.81 | 17 | 48 | 76 | 7 | 1 |
| lysosomal | 29.45 | 23 | 10 | 7 | Below | Below |
| protective | | | | | limit of | limit of |
| protein | | | | | quantitation | quantitation |
| glutathione | 24.04 | Below | Below | 1 | Below | Below |
| S-transferase | | limit of | limit of | | limit of | limit of |
| Y1 | | quantitation | quantitation | | quantitation | quantitation |
| 40S | −9.16 | 1 | 1 | Below | Below | Below |
| ribosomal | | | | limit of | limit of | limit of |
| protein S28 | | | | quantitation | quantitation | quantitation |
| thioredoxin | −15.94 | 3 | Below | 3 | 1 | Below |
| isoform X1 | | | limit of | | | limit of |
| | | | quantitation | | | quantitation |
| basement | −29.68 | 951 | Below | Below | Below | Below |
| membrane- | | | limit of | limit of | limit of | limit of |
| specific | | | quantitation | quantitation | quantitation | quantitation |
| heparan | | | | | | |
| sulfate | | | | | | |
| proteoglycan | | | | | | |
| core protein | | | | | | |
| isoform X1 | | | | | | |
| tubulointerstitial | −35.46 | 148 | Below | Below | Below | Below |
| nephritis | | | limit of | limit of | limit of | limit of |
| antigen-like | | | quantitation | quantitation | quantitation | quantitation |
| protein | | | | | | |
| actin - partial | −38.94 | 398 | Below | Below | Below | Below |
| cytoplasmic 2 | | | limit of | limit of | limit of | limit of |
| isoform X2 | | | quantitation | quantitation | quantitation | quantitation |
| galectin-1 | −45.49 | 14 | Below | Below | Below | Below |
| | | | limit of | limit of | limit of | limit of |
| | | | quantitation | quantitation | quantitation | quantitation |
| peroxiredoxin- | −50.43 | 86 | 9 | 13 | Below | Below |
| 1 | | | | | limit of | limit of |
| | | | | | quantitation | quantitation |
| cornifin alpha | −109.26 | 50 | 3 | 7 | Below | Below |
| | | | | | limit of | limit of |
| | | | | | quantitation | quantitation |

### Example 7. Impact of Depth Filter Type and Wi on HCP Reduction During Depth Filtration—mAb5 (Donanemab) and mAb6

### Part A—Impact of pH on HCP Reduction

Two antibodies (mAb5 and mAb6) are prepared using the protein capture step essentially as described for mAb1 in Example 1, except the elution step is performed with the buffer systems shown in Table 9. The total HCP content is measured by ELISA giving a range of about 2800 to about 3200 ppm. Following protein capture, the low pH viral inactivation step is performed essentially as described for mAb1 in Example 1, followed by a neutralization step at either pH 5.0 or pH 7.0 using 500 mM Tris base pH unadjusted buffer. The depth filtration step is performed essentially as described for mAb1 in Example 1 using a X0SP depth filter at a loading of 1000 g/m². The HCP content after the depth filtration step is measured by ELISA.

The results in Table 9 show significant reduction in total HCP content to less than ≤50 ppm for both antibodies following depth filtration when the pH of the mixture applied to the depth filter is pH 7.0. Total HCP content is reduced to a lesser extent when the pH of the mixture applied to the depth filter is pH 5.0.

TABLE 9
HCP levels in mAb5 (donanemab) and mAb6 preparations
following Protein A elution and depth filtration

| | | pH of material | HCP content |
|---|---|---|---|
| | | applied to | after depth |
| Antibody | Protein A elution buffer | depth filter | filtration |
| mAb5 | 20 mM acetic acid + 5 mM | pH 5 | 231 |
| (donanemab) | lactic acid | pH 7 | 45 |
| | 20 mM acetic acid + 5 mM | pH 5 | 229 |
| | phosphoric acid | pH 7 | 13 |
| mAb6 | 20 mM acetic acid + 5 mM | pH 5 | 338 |
| | lactic acid | pH 7 | 41 |
| | 20 mM acetic acid + 5 mM | pH 5 | 331 |
| | phosphoric acid | pH 7 | 9 |

Part B: Impact of Depth Filter and pH on HCP Reduction for mAb5

mAb5 is prepared using the protein capture step essentially as described in Example 5. The eluate is subjected to low pH viral inactivation and neutralization as essentially described in Example 5. For the depth filtration step, four different pH and depth filter set-ups were evaluated:
(i) B1HC filter+pH 5.1
(ii) X0SP filter+pH 5.1
(iii) X0SP filter+pH 6.2
(iv) X0SP filter+pH 7.3
   (i) B1HC filter+pH 5.1

mAb5 is prepared using the protein capture step essentially as described in Example 5. 500 mls is placed into glass beaker and mixed with a teflon stir bar. The protein concentration of the Protein A eluate is 12.5 mg/ml. With 500 mls in the beaker, the total protein content is 6250 mg (12.5 mg/ml×500 ml=6250 mg).

The starting pH of the solution in the beaker is 3.98 (temperature=18.1 C). The pH is adjusted to 3.45 with 20 mM acetic acid/5 mM citric acid to perform the low pH viral inactivation step as essentially described in Example 5.

While the low pH viral inactivation step is ongoing, a B1HC filter (micro pod or 23 sq cm, Lot CP7NA77798, part MB1HC23CL3) is set up. Size 14 platinum cured silicon tubing with PendoTech Filter Screening Peristaltic pumping system (K434694) with OHAUS Scout scales, K434696 to K434699) is used. All filters are flushed with PWTR at 23 ml/min (about 600 LMH) for 230 mls per filter or 100 L/sqm.

Neutralization to pH 5.0 is achieved with 0.25 M Tris base (EL19562-368, LB213, EXP 4/15/2020). The Solution turns cloudy as pH reaches 5 and the final pH is measured as 5.09 (5.1). The concentration is calculated to be 7.27 mg/ml (6250 mg/860 mls at pH 5). While stirring the pH 5 solution, filtration is begun through the B1HC filter with a load of 997 g/sqm (309 ml×7.27 mg/ml=2.246 g/0.0023 sqm=997 g/sqm. The B1HC filter is recovery flushed with 45 mls of PWTR. Filters are essentially pumped dry after recovery flush. The final volume of B1HC is 375.5 ml at 5.13 mg/ml providing a 85.8% yield of 1.926 g.

### (ii) X0SP Filter+pH 5.1 or pH 6.3 or pH 7.2

mAb5 is prepared using the protein capture step essentially as described in Example 5. 500 mls is placed into glass beaker and mixed with a Teflon stir bar. The protein concentration of the Protein A eluate is 15.75 mg/ml. With 500 mls in the beaker, the total protein content is 7875 mg (15.75 mg/ml×500 ml=7875 mg).

The starting pH of the solution in the beaker is 4.05 (temperature=18.1 C). The pH is adjusted to 3.45 with 20 mM acetic acid/5 mM citric acid to perform the low pH viral inactivation step as essentially described in Example 5.

While the low pH viral inactivation step is ongoing, a three X0SP filters (micro pod or 23 sq cm, Lot CP9AA93251, cat MX0SP23CL3) are setup and flushed separately as described above.

Neutralization I achieved with use 0.25 M Tris base (EL19562-368, LB213, EXP 4/15/2020):

A first beaker was pH adjusted to 5.1 with 20 mls of 250 mM Tris base. The calculated concentration is 9.04 mg/ml.

The second beaker was pH adjusted to 6.3 with 27 mls of 250 mM Tris base. The calculated concentration is 8.82 mg/ml.

The third beaker was pH adjusted to 7.2 with 32 mls of 250 mM Tris base. The calculated concentration is 8.67 mg/ml.

The precipitate for the pH 6.3 and 7.2 seemed slimy (as it would stick to bottom of glass towards end of filtration), and possibly larger in size than pH 5.1.

Filtration through the X0SP filters is begun while stirring the three solutions.

The pH 5.0 X0SP reached 25 psi at a load of 203 mls and then switching to water recovery flush. The load is calculated as 798 g/sqm (9.04 mg/ml×203 ml=1.835 g/0.0023 sq m=798 g/sq m).

Filters are recovery flushed with ˜45 mls of PWTR. Filters are essentially pumped dry after recovery flush.

Final Volume of X0SP pH 5.1=278 ml at 5.89 mg/ml=1.637 g Yield=1.637 g/1.835=89.2%

Final Volume of X0SP pH 6.3=365 ml at 5.76 mg/ml=1. g Yield=2.102 g/2.58=81.5%

Final Volume of X0SP pH 7.2=365 ml at 5.52 mg/ml=2.015 g/2.58=78.1%

(iii) AEX Chromatography

Each of the depth filtration preparations are subjected to AEX essentially as described in Example 5. For all AEX charge preparations, the filtrate at pH 5 and the filtrate at pH 6 (not the filtrate at 7.2) were pH adjusted to 7.25 with 250 mM Tris base (lot EL19562-368, LB213, exp 4-15-20, for development use) and then add NaCl to a final concentration of 50 mM using 20 mM Tris, 1 M NaCl, pH 7.0 (EL19562-862 LB198, exp 9-30-2020) at 0.0526×volume at pH 7.25. All charge preparations are performed in glass beaker with stir bar. 600 mg of each filtrate was used in order to load the AEX with the same amount. All AEX charge pHs were between 7.1 and 7.3, and all the conductivities were 6.5+/−0.2 mS.

Final AEX MS (at pH 5) volumes, mAb5 concentration, total mg, and yield were:
1. B1HC material—155 ml at 3.91 mg/ml=606.1 mg or 101%
2. X0SP at pH 5.1-120 ml at 5.00 mg/ml=600 mg or 100%
3. X0SP at pH 6.3-121 ml at 4.96 mg/ml=600.2 mg or 100%
4. X0SP at pH 7.2-126 ml at 4.79 mg/ml=603.5 mg or 100.6%
   (iii) CEX Chromatography

Each of the AEX preparations are subjected to CEX chromatography essentially as described in Example 5. The actual loads on the CEX resin are as follows:
(i) B1HC preparation at 3.91 mg/ml× volume loaded 130 ml×0.85=110.5 ml=432.1/17.28=25.0 mg/ml
(ii) X0SP at pH 5 at 5.00 mg/ml volume loaded 101.7 ml×0.85%=86.4 ml=432/17.28=25.0 mg/ml
(iii) X0SP at pH 6.3 at 4.96 mg/ml volume loaded=102.5×0.85=87.1 ml=432.0/17.28 ml=25.0 mg/ml
(iv) X0SP at pH 7.2 at 4.79 mg/ml volume loaded=106.1×0.85=90.2 ml=432.1/17.28=25.0 mg/ml

The CEX mainstream volumes, concentration and yields for each condition are as follows:
(i) B1HC at pH 5.0 at 5.83 mg/ml× MS volume=64.1 ml MS Volume=373 mg/432.1 mg=86.3%
(ii) X0SP at pH 5 at 5.83 mg/ml×64.8 ml MS Volume=377.8 mg/432 mg=87.5%
(iii) X0SP at pH 6.3 at 5.80 mg/ml=64.8 ml MS Volume=375.8 mg/432.0 mg=87.0%
(iv) X0SP at pH 7.2 at 5.80 mg/ml=64.7 ml MS Volume=375.3 mg/432.1 mg=86.9%

### (v) Analysis of HCP Content by LC-MS

The CEX preparations are analyzed for HCP content using LCM essentially as described in Example 5. The LC-MS data is provided in Table 10.

TABLE 10
Content of Host Cell Proteins in mAb5 (donanemab) Preparation After Protein Capture,
Low pH Viral Inactivation Step, Neutralization Step, and Depth Filtration

| | | Neutralization pH | | | | | | | |
|---|---|---|---|---|---|---|---|---|---|
| | | ~5.0 | | ~5.0 | | ~6.0 | | ~7.0 | |
| HCP ID | EpiMatrix | B1HC | | X0SP | | X0SP | | X0SP | |
| Total | N/A | 85.4 | ppm | 48.8 | ppm | 42.1 | ppm | 48.4 | ppm |
| protein S100-A6 | 52.84 | 0.3 | ppm | 0.2 | ppm | 0.1 | ppm | 0.1 | ppm |
| protein S100-A11 | 48.79 | 0.4 | ppm | 0.3 | ppm | 0.2 | ppm | 0.2 | ppm |
| phospholipase B- | 32.89 | 1.5 | ppm | 0.8 | ppm | 0.6 | ppm | 0.7 | ppm |
| like 2 protein | | | | | | | | | |
| lysosomal | 29.45 | 6.2 | ppm | 0.4 | ppm | 0.01 | ppm | 0.01 | ppm |
| protective protein | | | | | | | | | |
| ubiquitin-40S | 1.9 | 5.1 | ppm | 5.7 | ppm | 5.1 | ppm | 5.1 | ppm |
| ribosomal protein | | | | | | | | | |
| S27a | | | | | | | | | |
| Kallikrein-11 | −12.83 | — | | 2.7 | ppm | 0 | ppm | 0 | ppm |
| serine protease | −13 | 1.9 | ppm | 0.1 | ppm | ND or | | 0.0 | ppm |
| HTRA1 isoform | | | | | | below limit | | | |
| X1 | | | | | | of | | | |
| | | | | | | quantitation | | | |
| thioredoxin | −15.94 | 2.8 | ppm | 2.7 | ppm | 2.4 | ppm | 2.3 | ppm |
| isoform X1 | | | | | | | | | |
| complement C1r | −23.01 | 8.7 | ppm | 11.5 | ppm | 11.6 | ppm | 16.3 | ppm |
| subcomponent | | | | | | | | | |
| actin, aortic | −34.63 | 4.1 | ppm | 2.3 | ppm | 2.1 | ppm | 2.1 | ppm |
| smooth muscle | | | | | | | | | |
| isoform X1 | | | | | | | | | |
| galectin-1 | −45.49 | 0.4 | ppm | 0.4 | ppm | 0.3 | ppm | 0.5 | ppm |
| heat shock | −47.2 | 4.6 | ppm | 2.6 | ppm | 2.9 | ppm | 2.4 | ppm |
| cognate 71 kDa | | | | | | | | | |
| protein | | | | | | | | | |
| peroxiredoxin-1 | −50.43 | 21.7 | ppm | 8.3 | ppm | 4.3 | ppm | 4.2 | ppm |

| | | | | | | | | |
|---|---|---|---|---|---|---|---|---|
| cornifin alpha | −109.26 | 0.2 | ppm | 0.2 | ppm | ND or | 0.1 | ppm |
| | | | | | | below limit | | |
| | | | | | | of | | |
| | | | | | | quantitation | | |

The data in Table 10 show significant reduction in total HCP content to less than ≤50 ppm following depth filtration with the X0SP filter at all pHs tested. This compares favorably to the reduction in HCP content following depth filtration with the B1HC filter. It is also notable that the yield after the depth filtration step is lower at pH 6.3 and 7.2 in comparison to the lower pH 5.1. Therefore, the reduction in HCP content at high pH may be offset by the loss of yield. The optimal performance is seen with the X0SP filter at pH 5.0.

### Example 8. Method for Determination of Ionic Strength During Biomolecule Purification Processes

A method for the estimation of ionic strength based on what is known of the buffer compositions during biomolecule purification unit processes is herein described. The ionic strength (I) of a solution is a measure of concentration of ions in that solution, and is a function of species concentration, cᵢ, and net charge, zᵢ, for all species. To determine ionic strength, Formula I is used.

$$I = \frac{1}{2} {\sum }_{i} {c}_{i} {z}_{i}^{2} \qquad ( 1 )$$

Strong electrolytes: for strong electrolytes at low concentrations (e.g., below 50 mM), complete dissociation is assumed. With complete dissociation, the composition is easily calculated making ionic strength calculations straightforward. For example, a solution of 50 mM NaCl dissociates to give 50 mM each of Na⁺ and Cl⁻ with an ionic strength of 0.5×[50 mM× I²+50 mM× (−1)²]=50 mM. As another example, 50 mM Na₂SO₄ dissociates to give 100 mM of Na⁺ and 50 mM of SO₄²⁻, giving an ionic strength of 0.5×[100 mM× I²+50 mM× (−2)²]=150 mM. With no buffering species, near-neutral pH is expected in these calculations such that concentrations of ions from the dissociation of water do not contribute meaningfully to the ionic strength. The dissociation constant of water is taken to be K_{w}=[H⁺][OH⁻]=10⁻¹⁴ with [H⁺]=10^{-pH} where the square brackets indicate concentrations. For the purpose of calculations herein, physical interpretation of H⁺ ions (as opposed to hydronium ions, for example) is not necessary, and likewise it is not necessary to distinguish between H⁺ concentration and activity.
Buffered systems: for buffered systems complete dissociation cannot be assumed. Acid dissociation constants of the buffers must be used to determine the proportion of the buffer in the acid and base forms. For a generic acid, HA, that dissociates into H⁺ and A⁻ Formula 2 relates to the acid dissociation constant, Kₐ, and the species concentrations:

$${K}_{a} = \frac{[ {H}^{+} ] [ {A}^{-} ]}{[ H A ]} \qquad ( 2 )$$

The acid dissociation constant is often used in the logarithmic form of pKₐ=−log₁₀(Kₐ). The thermodynamic pKₐ, denoted as pK_{a,0}, is available in the literature for many buffers of interest. However, the effective pKₐ of a buffer diverges from the thermodynamic value except in very dilute solution due to deviation of activity coefficients from unity. For moderately dilute solutions considered in this disclosure, the extended Debye Hückel equation or Davies equation were used to account for non-unity activity coefficients. Values for some of the constants found in literature may differ slightly but give similar results in the range of ionic strength values of interest in the present disclosure. The extended Debye Hückel equation is provided as Formula 3:

$$p {K}_{a} = p {K}_{a , 0} + \frac{0.51 n \sqrt{I}}{1 + 1.6 \sqrt{I}} \qquad ( 3 )$$

The Davies equation is provided as Formula 4:

$$p {K}_{a} = p {K}_{a , 0} + 0 \text{.51} n ( \frac{\sqrt{I}}{1 + \sqrt{I}} - 0.3 \sqrt{I} ) \qquad ( 4 )$$

where n=2z−1 and z is the net charge of the acidic buffer form for calculating n (Scopes, Protein Purification: Principles and Practices, 2013).

Since pKₐ is a function of ionic strength, the composition and ionic strength cannot be determined independently, but are part of a system of equations. The system of equations includes the aforementioned equations for ionic strength, acid dissociation constants for each buffer, and pKₐ equations for each buffer, and also includes an electroneutrality condition and a total species balance for each buffer. With this system of equations, several values may be estimated. For example, a known solution pH can be used to estimate an acid-based ratio for a buffer formulation, or conversely an acid-based ratio can be used to estimate a solution pH and corresponding titration volumes. In any of these applications, the ionic strength can be estimated, to help guide rational selection of eluent and titrant options.

To calculate the ionic strength relevant to the buffered systems in the present disclosure, such as that of the feed material for depth filtration, the buffer composition of the solution is needed. This composition can be reasonably estimated based on the volumes and compositions of the buffers and titrants used in the process. Ion measurement techniques known in the field may also be used to estimate the composition.

As a starting point for estimating the solution composition, one possible methodology is to assume that the affinity column eluate pool has a buffer composition identical to that of the eluent with the exception of being buffered at the measured pH of the eluate pool. For example, if the protein of interest is eluted from a Protein A column with 20 mM acetic acid, 5 mM lactic acid and the eluate pool has a measured pH of 4.2, the assumption would be made that the buffer composition of the eluate pool is 20 mM acetate, 5 mM lactate, and sufficient NaOH to bring pH to 4.2; this would equate to about ˜8.2 mM NaOH. Because only the total sodium cation, Na⁺, content is important to the calculation, it does not matter whether the eluate sodium content is assumed to originate from sodium acetate, sodium phosphate, sodium hydroxide, or any combination thereof, so the convention of attributing the sodium to NaOH is used for convenience.

Having used the eluent composition and eluate pH to estimate the buffer composition of the eluate, the solution titrations are then considered. For example, with an estimated eluate composition of 20 mM acetate, 5 mM lactate, ˜8.2 mM NaOH at pH 4.2, if the volume of 20 mM HCl required to lower the pH to a target value of 3.45 for viral inactivation was equal to 0.305 times the start volume, then the composition of that process intermediate at pH 3.45 would be known from the dilution. Acetate, lactate, and NaOH would be present at 1/1.305 times their respective initial values (i.e., ˜15.3 mM acetate, ˜3.8 mM lactate, and ˜6.2 mM NaOH) and HCl present at 0.305/1.305 of its value in the titrant (˜4.7 mM HCl). Similarly, for neutralization with 250 mM Tris base, if the ratio to raise the pH to a target of pH 7.0 was 0.0743 times the volume of pH 3.45 solution, ratios of 1/1.0743 and 0.0743/1.0743 would be applied to find the final concentrations in the neutralized solution (˜14.3 mM acetate, ˜3.6 mM lactate, ˜5.8 mM NaOH, ˜4.4 mM HCl, and ˜17.3 mM Tris). All known values are plugged into the system of equations (Formulas 5 thru 15) to calculate the ionic strength:

$$I = \frac{1}{2} ( [ {H}^{+} ] \cdot \{ {1}^{2} \} + [ {Na}^{+} ] \cdot \{ {1}^{2} \} + [ {TrisH}^{+} ] \cdot \{ {1}^{2} \} + [ {OH}^{-} ] \cdot {\{ - 1 \}}^{2} + [ {Acetate}^{-} ] \cdot {\{ - 1 \}}^{2} + [ {Lactate}^{-} ] \cdot {\{ - 1 \}}^{2} + [ {Cl}^{-} ] \cdot {\{ - 1 \}}^{2} ) \qquad ( 5 )$$

$$[ {H}^{+} ] + [ {Na}^{+} ] + [ {TrisH}^{+} ] = [ {OH}^{-} ] + [ {Acetate}^{-} ] + [ {Acetate}^{-} ] + [ {Cl}^{-} ] \qquad ( 6 )$$

$${K}_{a , Tris} = \frac{[ {H}^{+} ] [ Tris ]}{[ {TrisH}^{+} ]} \qquad ( 7 )$$

$${pK}_{a , Tris} = {pK}_{a , 0 , Tris} + 0.51 ( 2 \cdot \{ + 1 \} - 1 ) ( \frac{\sqrt{I}}{1 + \sqrt{I}} - 0.3 \sqrt{I} ) \qquad ( 8 )$$

$${K}_{a , Acetate} = \frac{[ {H}^{+} ] [ {Acetate}^{-} ]}{[ H Acetate ]} \qquad ( 9 )$$

$${pK}_{a , Acetate} = {pK}_{a , 0 , Acetate} + 0.51 ( 2 \cdot \{ - 1 \} - 1 ) ( \frac{\sqrt{I}}{1 + \sqrt{I}} - 0.3 \sqrt{I} ) \qquad ( 10 )$$

$${K}_{a , Lactate} = \frac{[ {H}^{+} ] [ {Lactate}^{-} ]}{[ H Lactate ]} \qquad ( 11 )$$

$${pK}_{a , Lactate} = {pK}_{a , 0 , Lactate} + 0.51 ( 2 \cdot \{ - 1 \} - 1 ) ( \frac{\sqrt{I}}{1 + \sqrt{I}} - 0.3 \sqrt{I} ) \qquad ( 12 )$$

$$Total Tris = [ Tris ] + [ {TrisH}^{+} ] \qquad ( 13 )$$

$$Total Acetate = [ H Acetate ] + [ {Acetate}^{-} ] \qquad ( 14 )$$

$$Total Lactate = [ HLactate ] + [ {Lactate}^{-} ] \qquad ( 15 )$$

where respective pK_{a,0} value for Tris, acetate, and lactate were taken to be 8.15, 4.76, and 3.86 at 22° C. The resulting estimate for the ionic strength of the depth filtration feed 20 material is 22.1 mM.

As described herein, buffering capacity of a protein product is not directly modeled. Thus, when using a strong acid or base for titration, some deviations can arise between calculations and empirical titration results. For example, when titrating a Protein A eluate to low pH for viral inactivation, the buffer calculations typically underestimate 25 the empirical amount of 20 mM HCl needed; the empirical amount needed may be on the order of 50% greater than the calculated estimate. One way to account for this difference is to model the affinity column eluate material at a higher pH, empirically adjusting the value until the estimated titration volume matches the experimental value. For example, in the above example, if the amount of 20 mM HCl was 50% higher than the 0.305 ratio than initially estimated, the Protein A eluate would be modeled as being about pH 4.45 instead of pH 4.2. Making this empirical change to the modeling, the estimated ionic strength in the example is directionally reduced, but only by a small amount: 21.9 mM down from the initial 22.1 mM estimate. Accordingly, it is concluded that either approach is sufficient for estimating ionic strength to deduce preferred embodiments of the present disclosure.

Alternative methods: Ion content measurement methods can be used to determine the buffer composition of the depth filtration feed material to calculate the ionic strength. This requires confirming that the measurements give self-consistent results with any known amounts such as the amounts of titrant added. Since the buffer composition of the affinity column eluate is assumed to be equivalent to that of the eluent but at a different pH, the difference in true composition could be determined by ion content measurements. For example, either an amount based on the eluent composition, or a measured value may be used to calculate ionic strength of the buffer components in the eluent.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention.

### SEQUENCES

The following nucleic and/or amino acid sequences are referred to in the disclosure and are provided below for reference.

SEQ ID NO: 1-bamlanivimab variable heavy chain (VH)
QVQLVQSGAEVKKPGSSVKVSCKASGGTFSNYAISWVRQAPGQGLEWMGRIIPIL
GIANYAQKFQGRVTITADKSTSTAYMELSSLRSEDTAVYYCARGYYEARHYYYY
YAMDVWGQGTAVTVSS
SEQ ID NO: 2-bamlanivimab variable light chain (VL)
DIQMTQSPSSLSASVGDRVTITCRASQSISSYLSWYQQKPGKAPKLLIYAASSLQS
GVPSRFSGSGSGTDFTLTITSLQPEDFATYYCQQSYSTPRTFGQGTKVEIK
SEQ ID NO: 3-bamlanivimab heavy chain (HC)
QVQLVQSGAEVKKPGSSVKVSCKASGGTFSNYAISWVRQAPGQGLEWMGRIIPIL
GIANYAQKFQGRVTITADKSTSTAYMELSSLRSEDTAVYYCARGYYEARHYYYY
YAMDVWGQGTAVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTK
VDKRVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSV
MHEALHNHYTQKSLSLSPGK
SEQ ID NO: 4-bamlanivimab light chain (LC)
DIQMTQSPSSLSASVGDRVTITCRASQSISSYLSWYQQKPGKAPKLLIYAASSLQS
GVPSRFSGSGSGTDFTLTITSLQPEDFATYYCQQSYSTPRTFGQGTKVEIKRTVAA
PSVFIFPPSDEQLKSGTASVVCLLNNFYPREAKVQWKVDNALQSGNSQESVTEQD
SKDSTYSLSSTLTLSKADYEKHK VYACEVTHQGLSSPVTKSFNRGEC
SEQ ID NO: 5-etesevimab variable heavy chain (VH)
EVQLVESGGGLVQPGGSLRLSCAASGFTVSSNYMSWVRQAPGKGLEWVSVIYSG
GSTFYADSVKGRFTISRDNSMNTLFLQMNSLRAEDTAVYYCARVLPMYGDYLD
YWGQGTLVTVSS
SEQ ID NO: 6-etesevimab variable light chain (VL)
DIVMTQSPSSLSASVGDRVTITCRASQSISRYLNWYQQKPGKAPKLLIYAASSLQS
GVPSRFSGSGSGTDFTLTISSLQPEDFATYYCQQSYSTPPEYTFGQGTKLEIKRTV
SEQ ID NO: 7-etesevimab heavy chain (HC)
EVQLVESGGGLVQPGGSLRLSCAASGFTVSSNYMSWVRQAPGKGLEWVSVIYSG
GSTFYADSVKGRFTISRDNSMNTLFLQMNSLRAEDTAVYYCARVLPMYGDYLD
YWGQGTLVTVSSASTKGPSVFPLAPSSKSTSGGTAALGCLVKDYFPEPVTVSWNS
GALTSGVHTFPAVLQSSGLYSLSSVVTVPSSSLGTQTYICNVNHKPSNTKVDKRV
EPKSCDKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALH
NHYTQKSLSLSPGK
SEQ ID NO: 8-etesevimab light chain (LC)
DIVMTQSPSSLSASVGDRVTITCRASQSISRYLNWYQQKPGKAPKLLIYAASSLQS
GVPSRFSGSGSGTDFTLTISSLQPEDFATYYCQQSYSTPPEYTFGQGTKLEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 9-bebtelovimab variable heavy chain (VH)
QITLKESGPTLVKPTQTLTLTCTFSGFSLSISGVGVGWLRQPPGKALEWLALIYWD
DDKRYSPSLKSRLTISKDTSKNQVVLKMTNIDPVDTATYYCAHHSISTIFDHWGQ
GTLVTVSS
SEQ ID NO: 10-bebtelovimab variable light chain (VL)
QSALTQPASVSGSPGQSITISCTATSSDVGDYNYVSWYQQHPGKAPKLMIFEVSD
RPSGISNRFSGSKSGNTASLTISGLQAEDEADYYCSSYTTSSAVFGGGTKLTVL
SEQ ID NO: 11-bebtelovimab heavy chain (HC)
QITLKESGPTLVKPTQTLTLTCTFSGFSLSISGVGVGWLRQPPGKALEWLALIYWD
DDKRYSPSLKSRLTISKDTSKNQVVLKMTNIDPVDTATYYCAHHSISTIFDHWGQ
GTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKRVEPKSC
DKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYT
QKSLSLSPGK
SEQ ID NO: 12-bebtelovimab light chain (LC)
QSALTQPASVSGSPGQSITISCTATSSDVGDYNYVSWYQQHPGKAPKLMIFEVSD
RPSGISNRFSGSKSGNTASLTISGLQAEDEADYYCSSYTTSSAVFGGGTKLTVLGQ
PKAAPSVTLFPPSSEELQANKATLVCLISDFYPGAVTVAWKADSSPVKAGVETTT
PSKQSNNKYAASSYLSLTPEQWKSHRSYSCQVTHEGSTVEKTVAPTECS
SEQ ID NO: 13-LCVR of Donanemab
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
K
SEQ ID NO: 14-HCVR of Donanemab
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSS
SEQ ID NO: 15-LC of Donanemab
DIVMTQTPLSLSVTPGQPASISCKSSQSLLYSRGKTYLNWLLQKPGQSPQLLIYAV
SKLDSGVPDRFSGSGSGTDFTLKISRVEAEDVGVYYCVQGTHYPFTFGQGTKLEI
KRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC
SEQ ID NO: 16-HC of Donanemab
QVQLVQSGAEVKKPGSSVKVSCKASGYDFTRYYINWVRQAPGQGLEWMGWINP
GSGNTKYNEKFKGRVTITADESTSTAYMELSSLRSEDTAVYYCAREGITVYWGQ
GTTVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHY
TQKSLSLSPG
SEQ ID NO: 17-LCDR1 of Donanemab
KSSQSLLYSRGKTYLN
SEQ ID NO: 18-LCDR2 of Donanemab
AVSKLDS
SEQ ID NO: 19-LCDR3 of Donanemab
VQGTHYPFT
SEQ ID NO: 20-HCDR1 of Donanemab
GYDFTRYYIN
SEQ ID NO: 21-HCDR2 of Donanemab
WINPGSGNTKYNEKFKG
SEQ ID NO: 22-HCDR3 of Donanemab
EGITVY
SEQ ID NO: 23-LCVR of Antibody 201c (mAb7)
DIQMTQSPSTLSASVGDRVTITCRASQSLGNWLAWYQQKPGKAPKLLIYQASTLE
SGVPSRFSGSGSGTEFTLTISSLQPDDFATYYCQHYKGSFWTFGQGTKVEIK
SEQ ID NO: 24-HCVR of Antibody 201c (mAb7)
EVQLLESGGGLVQPGGSLRLSCAASGFTFSSYPMSWVRQAPGKGLEWVSAISGS
GGSTYYADSVKGRFTISRDNSKNTLYLQMNSLRAEDTAVYYCAREGGSGSYYN
GFDYWGQGTLVTVSS
SEQ ID NO: 25-LC of Antibody 201c (mAb7)
DIQMTQSPSTLSASVGDRVTITCRASQSLGNWLAWYQQKPGKAPKLLIYQASTLE
SGVPSRFSGSGSGTEFTLTISSLQPDDFATYYCQHYKGSFWTFGQGTKVEIKRTVA
APSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 26-HC of Antibody 201c (mAb7)
EVQLLESGGGLVQPGGSLRLSCAASGFTFSSYPMSWVRQAPGKGLEWVSAISGS
GGSTYYADSVKGRFTISRDNSKNTLYLQMNSLRAEDTA VYYCAREGGSGSYYN
GFDYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSCSVMHE
ALHNHYTQKSLSLSPG
SEQ ID NO: 27-LCDR1 of Antibody 201c (mAb7)
RASQSLGNWLA
SEQ ID NO: 28-LCDR2 of Antibody 201c (mAb7)
YQASTLES
SEQ ID NO: 29-LCDR3 of Antibody 201c (mAb7)
QHYKGSFWT
SEQ ID NO: 30-HCDR1 of Antibody 201c (mAb7)
AASGFTFSSYPMS
SEQ ID NO: 31-HCDR2 of Antibody 201c (mAb7)
AISGSGGSTYYADSVKG
SEQ ID NO: 32-HCDR3 of Antibody 201c (mAb7)
AREGGSGSYYNGFDY
SEQ ID NO: 33-LC DNA sequence of Donanemab
gatattgtgatgactcagactccactctccctgtccgtcacccctggacagccggcctccatctcctgcaagtcaagtcagagcct
cttatatagtcgcggaaaaacctatttgaattggctcctgcagaagccaggccaatctccacagctcctaatttatgcggtgtctaaa
ctggactctggggtcccagacagattcagcggcagtgggtcaggcacagatttcacactgaaaatcagcagggtggaggccga
agatgttggggtttattactgcgtgcaaggtacacattacccattcacgtttggccaagggaccaagctggagatcaaacgaactg
tggctgcaccatctgtcttcatcttcccgccatctgatgagcagttgaaatctggaactgcctctgttgtgtgcctgctgaataacttct
atcccagagaggccaaagtacagtggaaggtggataacgccctccaatcgggtaactcccaggagagtgtcacagagcagga
cagcaaggacagcacctacagcctcagcagcaccctgacgctgagcaaagcagactacgagaaacacaaagtctacgcctgc
gaagtcacccatcagggcctgagctcgcccgtcacaaagagcttcaacaggggagagtgc
SEQ ID NO: 34-HC DNA Sequence of Donanemab
caggtgcagctggtgcagtctggggctgaggtgaagaagcctgggtcctcagtgaaggtttcctgcaaggcatctggttacgac
ttcactagatactatataaactgggtgcgacaggcccctggacaagggcttgagtggatgggatggattaatcctggaagcggta
atactaagtacaatgagaaattcaagggcagagtcaccattaccgcggacgaatccacgagcacagcctacatggagctgagc
agcctgagatctgaggacacggccgtgtattactgtgcgagagaaggcatcacggtctactggggccaagggaccacggtcac
cgtctcctcagcctccaccaagggcccatcggtcttcccgctagcaccctcctccaagagcacctctgggggcacagcggccct
gggctgcctggtcaaggactacttccccgaaccggtgacggtgtcgtggaactcaggcgccctgaccagcggcgtgcacacct
tcccggctgtcctacagtcctcaggactctactccctcagcagcgtggtgaccgtgccctccagcagcttgggcacccagaccta
catctgcaacgtgaatcacaagcccagcaacaccaaggtggacaagaaagttgagcccaaatcttgtgacaaaactcacacatg
cccaccgtgcccagcacctgaactcctggggggaccgtcagtcttcctcttccccccaaaacccaaggacaccctcatgatctcc
cggacccctgaggtcacatgcgtggtggtggacgtgagccacgaagaccctgaggtcaagttcaactggtacgtggacggcgt
ggaggtgcataatgccaagacaaagccgcgggaggagcagtacaacagcacgtaccgtgtggtcagcgtcctcaccgtcctg
caccaggactggctgaatggcaaggagtacaagtgcaaggtctccaacaaagccctcccagcccccatcgagaaaaccatctc
caaagccaaagggcagccccgagaaccacaggtgtacaccctgcccccatcccgggacgagctgaccaagaaccaggtca
gcctgacctgcctggtcaaaggcttctatcccagcgacatcgccgtggagtgggagagcaatgggcagccggagaacaacta
caagaccacgccccccgtgctggactccgacggctccttcttcctctatagcaagctcaccgtggacaagagcaggtggcagc
aggggaacgtcttctcatgctccgtgatgcatgaggctctgcacaaccactacacgcagaagagcctctccctgtctccgggt
SEQ ID NO: 35-LC DNA Sequence of Antibody 201c
gacatccagatgacccagtctccttccaccctgtctgcatctgtaggagacagagtcaccatcacttgccgggccagtcagagtct
tggtaactggttggcctggtatcagcagaaaccagggaaagcccctaaactcctgatctatcaggcgtctactttagaatctgggg
tcccatcaagattcagcggcagtggatctgggacagagttcactctcaccatcagcagcctgcagcctgatgattttgcaacttatt
actgccaacattataaaggttctttttggacgttcggccaagggaccaaggtggaaatcaaacggaccgtggctgcaccatctgtc
ttcatcttcccgccatctgatgagcagttgaaatctggaactgcctctgttgtgtgcctgctgaataacttctatcccagagaggcca
aagtacagtggaaggtggataacgccctccaatcgggtaactcccaggagagtgtcacagagcaggacagcaaggacagca
cctacagcctcagcagcaccctgacgctgagcaaagcagactacgagaaacacaaagtctacgcctgcgaagtcacccatca
gggcctgagctcgcccgtcacaaagagcttcaacaggggagagtgc
SEQ ID NO: 36-HC DNA Sequence of Antibody 201c
gaggtgcagctgttggagtctgggggaggcttggtacagcctggggggtccctgagactctcctgtgcagcctctggattcacct
ttagcagctatcctatgagctgggtccgccaggctccagggaaggggctggagtgggtctcagctattagtggtagtggtggtag
cacatactacgcagactccgtgaagggccggttcaccatctccagagacaattccaagaacacgctgtatctgcaaatgaacag
cctgagagccgaggacacggccgtatattactgtgcgagagaggggggctcagggagttattataacggctttgattattgggg
ccagggaaccctggtcaccgtctcctcagcctccaccaagggcccatcggtcttcccgctagcaccctcctccaagagcacctc
tgggggcacagcggccctgggctgcctggtcaaggactacttccccgaaccggtgacggtgtcgtggaactcaggcgccctg
accagcggcgtgcacaccttcccggctgtcctacagtcctcaggactctactccctcagcagcgtggtgaccgtgccctccagc
agcttgggcacccagacctacatctgcaacgtgaatcacaagcccagcaacaccaaggggacaagaaagttgagcccaaatc
ttgtgacaaaactcacacatgcccaccgtgcccagcacctgaactcctggggggaccgtcagtcttcctcttccccccaaaaccc
aaggacaccctcatgatctcccggacccctgaggtcacatgcgtggtggtggacgtgagccacgaagaccctgaggtcaagtt
caactggtacgtggacggcgtggaggtgcataatgccaagacaaagccgcgggaggagcagtacaacagcacgtaccgtgt
ggtcagcgtcctcaccgtcctgcaccaggactggctgaatggcaaggagtacaagtgcaaggtctccaacaaagccctcccag
cccccatcgagaaaaccatctccaaagccaaagggcagccccgagaaccacaggtgtacaccctgcccccatcccgggacg
agctgaccaagaaccaggtcagcctgacctgcctggtcaaaggcttctatcccagcgacatcgccgtggagtgggagagcaat
gggcagccggagaacaactacaagaccacgccccccgtgctggactccgacggctccttcttcctctatagcaagctcaccgtg
gacaagagcaggtggcagcaggggaacgtcttctcatgctccgtgatgcatgaggctctgcacaaccactacacgcagaagag
cctctccctgtctccgggt

1-172. (canceled)

173. A pharmaceutical composition comprising an antibody that binds to human N3pGlu Aβ (anti-N3pGlu Aβ antibody), wherein the anti-N3pGlu Aβ antibody was prepared by a process comprising purifying the anti-N3pGlu antibody from a mammalian host cell, and wherein the total content of host cell proteins (HCPs) in the composition is less than about 100 ppm (as measured by LCMS) and the composition comprises one of, combinations of, or all of the following host cell proteins: protein S100-A6, protein S100-A11, phospholipase B-like 2 protein, lysosomal protective protein, ubiquitin-40S ribosomal protein S27a, kallikrein-11, serine protease HTRA1 isoform X1, complement C1r subcomponent, actin, aortic smooth muscle isoform X1, heat shock cognate 71 kDa protein, and peroxiredoxin-1.

174. A pharmaceutical composition according to claim 173, wherein the mammalian cell is a CHO cell.

175. A pharmaceutical composition according to claim 173, wherein the anti-N3pGlu Aβ antibody is a monoclonal antibody, a chimeric antibody, a humanized antibody, a human antibody, a bispecific antibody, or an antibody fragment.

176. A pharmaceutical composition according to claim 175, wherein the anti-N3pGlu Aβ antibody is an IgG1 antibody.

177. A pharmaceutical composition according to claim 173, wherein the anti-N3pGlu Aβ antibody comprises a heavy chain (HC) and a light chain (LC), wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is KSSQSLLYSRGKTYLN (SEQ ID NO:17), LCDR2 is AVSKLDS (SEQ ID NO:18), LCDR3 is VQGTHYPFT (SEQ ID NO:19), HCDR1 is GYDFTRYYIN (SEQ ID NO:20), HCDR2 is WINPGSGNTKYNEKFKG (SEQ ID NO:21), and HCDR3 is EGITVY (SEQ ID NO:22).

178. A pharmaceutical composition according to claim 173, wherein the LC of the anti-N3pGlu Aβ antibody comprises a LCVR and the HC of the anti-N3pGlu Aβ antibody comprises a HCVR, wherein the LCVR is
(SEQ ID NO: 13)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIK
and the HCVR is
(SEQ ID NO: 14)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSS

179. A pharmaceutical composition according claim 173, wherein the LC of the anti-N3pGlu Aβ antibody is
(SEQ ID NO: 15)
DIVMTQTPLSLSVTPGQPASISCKSSQSLLYSRGKTYLNWLLQKPGQSP
QLLIYAVSKLDSGVPDRFSGSGSGTDFTLKISRVEAEDVGVYYCVQGTH
YPFTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
and the HC of the anti-N3pGlu Aβ antibody is
(SEQ ID NO: 16)
QVQLVQSGAEVKKPGSSVKVSCKASGYDFTRYYINWVRQAPGQGLEWMG
WINPGSGNTKYNEKFKGRVTITADESTSTAYMELSSLRSEDTAVYYCAR
EGITVYWGQGTTVTVSSASTKGPSVFPLAPSSKSTSGGTAALGCLVKDY
FPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSL
SPG.

180. A pharmaceutical composition according to claim 173, wherein the anti-N3pGlu Aβ antibody is donanemab.

181. (canceled)

182. A pharmaceutical composition according to claim 173, wherein the composition comprises less than about 5 ppm of protein S100-A6 (as measured by LCMS).

183. A pharmaceutical composition according to claim 173, wherein the composition comprises less than about 5 ppm of protein S100-A11 (as measured by LCMS).

184. A pharmaceutical composition according to claim 173, wherein the composition comprises less than about 10 ppm of phospholipase B-like 2 protein (as measured by LCMS).

185. A pharmaceutical composition according to claim 173, wherein the composition comprises less than about 5 ppm of lysosomal protective protein (as measured by LCMS).

186. A pharmaceutical composition according to claim 173, wherein the composition comprises less than about 5 ppm of ubiquitin-40S ribosomal protein S27a (as measured by LCMS).

187. A pharmaceutical composition according to claim 173, wherein the composition comprises less than about 5 ppm of kallikrein-11 (as measured by LCMS).

188. A pharmaceutical composition according to claim 173, wherein the composition comprises less than about 5 ppm serine protease HTRA1 isoform X1 (as measured by LCMS).

189. A pharmaceutical composition according to claim 173, wherein the composition comprises less than about 5 ppm complement C1r subcomponent (as measured by LCMS).

190. A pharmaceutical composition according to claim 173, wherein the composition comprises less than about 5 ppm actin (as measured by LCMS).

191. A pharmaceutical composition according to claim 173, wherein the composition comprises less than about 5 ppm aortic smooth muscle isoform X1 (as measured by LCMS).

192. A pharmaceutical composition according to claim 173, wherein the composition comprises less than about 5 ppm heat shock cognate 71 kDa protein (as measured by LCMS).

193. A pharmaceutical composition according to claim 173, wherein the composition comprises less than about 5 ppm peroxiredoxin-1 (as measured by LCMS).

194-196. (canceled)

197. A pharmaceutical composition comprising an antibody that binds to human N3pGlu Ab (anti-N3pGlu Ab antibody), wherein the anti-N3pGlu Ab antibody was prepared by a process comprising purifying the anti-N3pGlu antibody from a mammalian host cell, and wherein the total content of host cell proteins (HCPs) in the composition is less than about 10 ppm (as measured by LCMS) and the composition comprises one of, combinations of, or all of the following host cell proteins: polyubiquitin, lysosomal protective protein, glutathione S-transferase Y1, 40S ribosomal protein S28, thioredoxin isoform X1, basement membrane-specific heparan sulfate proteoglycan core protein isoform X1, tubulointerstitial nephritis antigen-like protein, actin-partial cytoplasmic 2 isoform X2, galectin-1, peroxiredoxin-1, and cornifin alpha.

198. (canceled)

199. A pharmaceutical composition according to claim 197, wherein the composition comprises less than about 1 ppm of polyubiquitin (as measured by LCMS).

200. A pharmaceutical composition according to claim 197, wherein the composition comprises less than about 1 ppm of lysosomal protective protein (as measured by LCMS).

201. A pharmaceutical composition according to claim 197, wherein the composition comprises less than about 1 ppm of glutathione S-transferase Y1 (as measured by LCMS).

202. (canceled)

203. A pharmaceutical composition according to claim 197, wherein the composition comprises less than about 1 ppm of 40S ribosomal protein S28 (as measured by LCMS).

204. A pharmaceutical composition according to claim 197, wherein the composition comprises less than about 1 ppm of thioredoxin isoform X1 (as measured by LCMS).

205. A pharmaceutical composition according to claim 197, wherein the composition comprises less than about 1 ppm of basement membrane-specific heparan sulfate proteoglycan core protein isoform X1 (as measured by LCMS).

206. A pharmaceutical composition according to claim 197, wherein the composition comprises less than about 1 ppm of tubulointerstitial nephritis antigen-like protein (as measured by LCMS).

207. A pharmaceutical composition according to claim 197, wherein the composition comprises less than about 1 ppm of actin-partial cytoplasmic 2 isoform X2 (as measured by LCMS).

208. A pharmaceutical composition according to claim 197, wherein the composition comprises less than about 1 ppm of galectin-1 (as measured by LCMS).

209. A pharmaceutical composition according to claim 197, wherein the composition comprises less than about 1 ppm of peroxiredoxin-1 (as measured by LCMS).

210. A pharmaceutical composition according to claim 197, wherein the mammalian cell is a CHO cell.

211. A pharmaceutical composition according to claim 197, wherein the anti-N3pGlu Aβ antibody is a monoclonal antibody, a chimeric antibody, a humanized antibody, a human antibody, a bispecific antibody, or an antibody fragment.

212. A pharmaceutical composition according to claim 197, wherein the anti-N3pGlu Aβ antibody is an IgG1 antibody.

213. The pharmaceutical composition according to claim 197, wherein the anti-N3pGlu Aβ antibody comprises a heavy chain (HC) and a light chain (LC), wherein the light chain comprises a light chain variable region (LCVR) and the heavy chain comprises a heavy chain variable region (HCVR), wherein the LCVR comprises amino acid sequences LCDR1, LCDR2, and LCDR3, and the HCVR comprises amino acid sequences HCDR1, HCDR2, and HCDR3, wherein LCDR1 is RASQSLGNWLA (SEQ ID NO: 27), LCDR2 is YQASTLES (SEQ ID NO: 28). LCDR3 is QHYKGSFWT (SEQ ID NO: 29), HCDR1 is AASGFTFSSYPMS (SEQ ID NO: 30), HCDR2 is AISGSGGSTYYADSVKG (SEQ ID NO: 31), and HCDR3 is AREGGSGSYYNGFDY (SEQ ID NO: 32).

214. The pharmaceutical composition of claim 197, wherein the LC of the anti-N3pGlu Aβ antibody comprises a LCVR and the HC of the anti-N3pGlu Aβ antibody comprises a HCVR, wherein the LCVR is
(SEQ ID NO: 23)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIK
and the HCVR is
(SEQ ID NO: 24)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSS.

215. The pharmaceutical composition of claim 197, wherein the LC of the anti-N3pGlu Aβ antibody is
(SEQ ID NO: 25)
DIQMTQSPSTLSASVGDRVTITCRASQSLGNWLAWYQQKPGKAPKLLIY
QASTLESGVPSRFSGSGSGTEFTLTISSLQPDDFATYYCQHYKGSFWTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
and the HC of the anti-N3pGlu Aβ antibody is
(SEQ ID NO: 26)
EVQLLESGGGLVQPGGSLRLSCAASGFTFSSYPMSWVRQAPGKGLEWVS
AISGSGGSTYYADSVKGRFTISRDNSKNTLYLQMNSLRAEDTAVYYCAR
EGGSGSYYNGFDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPG.


SEQUENCE LISTING (36 sequences)

SEQ ID NO: 1 - protein, 125 aa, Artificial Sequence
  Synthetic Construct
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSS

SEQ ID NO: 2 - protein, 107 aa, Artificial Sequence
  Synthetic Construct
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIK

SEQ ID NO: 3 - protein, 455 aa, Artificial Sequence
  Synthetic Construct
QVQLVQSGAEVKKPGSSVKVSCKASGGTFSNYAISWVRQAPGQGLEWMGRIIPILGIANYAQKFQGRVTITADKSTSTAYMELSSLRSEDTAVYYCARGYYEARHYYYYYAMDVWGQGTAVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  Synthetic Construct
DIQMTQSPSSLSASVGDRVTITCRASQSISSYLSWYQQKPGKAPKLLIYAASSLQSGVPSRFSGSGSGTDFTLTITSLQPEDFATYYCQQSYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 119 aa, Artificial Sequence
  Synthetic Construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSS

SEQ ID NO: 6 - protein, 112 aa, Artificial Sequence
  Synthetic Construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTV

SEQ ID NO: 7 - protein, 449 aa, Artificial Sequence
  Synthetic Construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 216 aa, Artificial Sequence
  Synthetic Construct
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 119 aa, Artificial Sequence
  Synthetic Construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSS

SEQ ID NO: 10 - protein, 109 aa, Artificial Sequence
  Synthetic Construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVL

SEQ ID NO: 11 - protein, 449 aa, Artificial Sequence
  Synthetic Construct
QITLKESGPTLVKPTQTLTLTCTFSGFSLSISGVGVGWLRQPPGKALEWLALIYWDDDKRYSPSLKSRLTISKDTSKNQVVLKMTNIDPVDTATYYCAHHSISTIFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 215 aa, Artificial Sequence
  Synthetic Construct
QSALTQPASVSGSPGQSITISCTATSSDVGDYNYVSWYQQHPGKAPKLMIFEVSDRPSGISNRFSGSKSGNTASLTISGLQAEDEADYYCSSYTTSSAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 112 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 14 - protein, 115 aa, Artificial Sequence
  Synthetic Construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 15 - protein, 219 aa, Artificial Sequence
  Synthetic Construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 444 aa, Artificial Sequence
  Synthetic Construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 16 aa, Artificial Sequence
  Synthetic Construct
KSSQSLLYSRGKTYLN

SEQ ID NO: 18 - protein, 7 aa, Artificial Sequence
  Synthetic Construct
AVSKLDS

SEQ ID NO: 19 - protein, 9 aa, Artificial Sequence
  Synthetic Construct
VQGTHYPFT

SEQ ID NO: 20 - protein, 10 aa, Artificial Sequence
  Synthetic Construct
GYDFTRYYIN

SEQ ID NO: 21 - protein, 17 aa, Artificial Sequence
  Synthetic Construct
WINPGSGNTKYNEKFKG

SEQ ID NO: 22 - protein, 6 aa, Artificial Sequence
  Synthetic Construct
EGITVY

SEQ ID NO: 23 - protein, 107 aa, Artificial Sequence
  Synthetic Construct
DIQMTQSPSTLSASVGDRVTITCRASQSLGNWLAWYQQKPGKAPKLLIYQASTLESGVPSRFSGSGSGTEFTLTISSLQPDDFATYYCQHYKGSFWTFGQGTKVEIK

SEQ ID NO: 24 - protein, 122 aa, Artificial Sequence
  Synthetic Construct
EVQLLESGGGLVQPGGSLRLSCAASGFTFSSYPMSWVRQAPGKGLEWVSAISGSGGSTYYADSVKGRFTISRDNSKNTLYLQMNSLRAEDTAVYYCAREGGSGSYYNGFDYWGQGTLVTVSS

SEQ ID NO: 25 - protein, 214 aa, Artificial Sequence
  Synthetic Construct
DIQMTQSPSTLSASVGDRVTITCRASQSLGNWLAWYQQKPGKAPKLLIYQASTLESGVPSRFSGSGSGTEFTLTISSLQPDDFATYYCQHYKGSFWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 451 aa, Artificial Sequence
  Synthetic Construct
EVQLLESGGGLVQPGGSLRLSCAASGFTFSSYPMSWVRQAPGKGLEWVSAISGSGGSTYYADSVKGRFTISRDNSKNTLYLQMNSLRAEDTAVYYCAREGGSGSYYNGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 11 aa, Artificial Sequence
  Synthetic Construct
RASQSLGNWLA

SEQ ID NO: 28 - protein, 8 aa, Artificial Sequence
  Synthetic Construct
YQASTLES

SEQ ID NO: 29 - protein, 9 aa, Artificial Sequence
  Synthetic Construct
QHYKGSFWT

SEQ ID NO: 30 - protein, 13 aa, Artificial Sequence
  Synthetic Construct
AASGFTFSSYPMS

SEQ ID NO: 31 - protein, 17 aa, Artificial Sequence
  Synthetic Construct
AISGSGGSTYYADSVKG

SEQ ID NO: 32 - protein, 15 aa, Artificial Sequence
  Synthetic Construct
AREGGSGSYYNGFDY

SEQ ID NO: 33 - DNA, 657 bp, Artificial Sequence
  Synthetic Construct
gatattgtgatgactcagactccactctccctgtccgtcacccctggacagccggcctccatctcctgcaagtcaagtcagagcctcttatatagtcgcggaaaaacctatttgaattggctcctgcagaagccaggccaatctccacagctcctaatttatgcggtgtctaaactggactctggggtcccagacagattcagcggcagtgggtcaggcacagatttcacactgaaaatcagcagggtggaggccgaagatgttggggtttattactgcgtgcaaggtacacattacccattcacgtttggccaagggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 34 - DNA, 1332 bp, Artificial Sequence
  Synthetic Construct
caggtgcagctggtgcagtctggggctgaggtgaagaagcctgggtcctcagtgaaggtttcctgcaaggcatctggttacgacttcactagatactatataaactgggtgcgacaggcccctggacaagggcttgagtggatgggatggattaatcctggaagcggtaatactaagtacaatgagaaattcaagggcagagtcaccattaccgcggacgaatccacgagcacagcctacatggagctgagcagcctgagatctgaggacacggccgtgtattactgtgcgagagaaggcatcacggtctactggggccaagggaccacggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 35 - DNA, 642 bp, Artificial Sequence
  Synthetic Construct
gacatccagatgacccagtctccttccaccctgtctgcatctgtaggagacagagtcaccatcacttgccgggccagtcagagtcttggtaactggttggcctggtatcagcagaaaccagggaaagcccctaaactcctgatctatcaggcgtctactttagaatctggggtcccatcaagattcagcggcagtggatctgggacagagttcactctcaccatcagcagcctgcagcctgatgattttgcaacttattactgccaacattataaaggttctttttggacgttcggccaagggaccaaggtggaaatcaaacggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgc

SEQ ID NO: 36 - DNA, 1353 bp, Artificial Sequence
  Synthetic Construct
gaggtgcagctgttggagtctgggggaggcttggtacagcctggggggtccctgagactctcctgtgcagcctctggattcacctttagcagctatcctatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagagaggggggctcagggagttattataacggctttgattattggggccagggaaccctggtcaccgtctcctcagcctccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt